(19) United States
(12) Patent Application Publication
(10) Pub. No.: US 2022/0233713 A1
(43) Pub. Date: Jul. 28, 2022

(54) SONODYNAMIC THERAPY

(71) Applicants: John CALLAN, Newtownabbey Antrim (GB); Anthony MCHALE, Newtownabbey Antrim (GB); Keiran LOGAN, Newtownabbey Antrim (GB)

(72) Inventors: John CALLAN, Newtownabbey Antrim (GB); Anthony MCHALE, Newtownabbey Antrim (GB); Keiran LOGAN, Newtownabbey Antrim (GB)

(21) Appl. No.: 17/617,701

(22) PCT Filed: Jun. 11, 2020
(86) PCT No.: PCT/GB2020/051411

§ 371 Date: Dec. 9, 2021

(30) Foreign Application Priority Data
Jun. 11, 2019 (GB) 1908352.6

(51) Int. Cl. A61K 47/69; A61K 41/00; A61K 45/06; A61K 47/24; A61K 47/54; A61K 9/00; A61K 31/7068

(52) U.S. Cl. CPC A61K 47/6925; A61K 41/0033; A61K 45/06; A61K 31/7068; A61K 47/544; A61K 9/0009; A61K 47/24

(57) ABSTRACT

The invention provides a method of preparing a microbubble covalently attached to at least one therapeutic agent which comprises: (i) providing a lipid (e.g. a phospholipid) capable of forming a microbubble; covalently linking at least one therapeutic agent to said lipid to produce a functionalised lipid; and preparing a microbubble from said functionalised lipid. Microbubble-therapeutic agent complexes which comprise a microbubble shell formed from a plurality of lipids (e.g. phospholipids) in which at least a proportion of the lipids are covalently linked to at least one therapeutic agent are also provided. Examples of therapeutic agents which may be attached to the microbubble include chemotherapeutic agents and sonosensitising agents. The complexes find use in methods of sonodynamic therapy and, in particular, in methods of combined sonodynamic therapy and chemotherapy.

### TECHNICAL FIELD

The present invention generally relates to sonodynamic therapy using microbubbles to deliver a therapeutic agent, or combination of therapeutic agents, to cells or tissues of interest, either in vitro or in vivo. In particular, it relates to a combined sonodynamic and anti-cancer therapy.

More specifically, the invention relates to improved methods for the preparation of microbubbles in which at least one therapeutic agent, such as a chemotherapeutic or sonosensitising agent, is covalently attached to the lipid shell of the microbubble.

The invention also relates to microbubble-complexes produced by such methods, to pharmaceutical compositions containing them, and to their use in methods of sonodynamic therapy, for example in the treatment of deeply-sited tumours such as pancreatic cancer. More particularly, it relates to the use of such microbubble-complexes in a combination therapy in which sonodynamic treatment is combined with treatment with at least one chemotherapeutic agent.

### BACKGROUND OF THE INVENTION

Conventional treatment of deeply-sited tumours typically involves major surgery, chemotherapy, radiotherapy or combinations of all of these. All three interventions may result in various complications including sepsis. Therefore, the development of more targeted and less invasive therapeutic approaches with improved efficacy to treat such patients is highly sought after. Pancreatic cancer is one example of a deeply-sited tumour. It remains one of the most lethal types of cancer known with less than 20% of those diagnosed being eligible for curative surgical treatment. It accounts for approximately 2% of all cancers with a five year survival of 15-21% in patients who have a surgical resection followed by systemic chemotherapy.

Methods known for use in the treatment of cancer include photodynamic therapy (PDT). PDT involves the application of photosensitising agents to the affected area, followed by exposure to photoactivating light to convert these into cytotoxic form. This results in the destruction of cells and surrounding vasculature in a target tissue. Photosensitisers which are currently approved for use in PDT absorb light in the visible region (below 700 nm). However, light of this wavelength has limited ability to penetrate the skin; this penetrates to a surface depth of only a few mm. Whilst PDT may be used to treat deeper sited target cells, this generally involves the use of a device, such as a catheter-directed fibre optic, for activation of the photosensitiser. Not only is this a complicated procedure, but it precludes access to certain areas of the body. It also compromises the non-invasive nature of the treatment. Thus, although appropriate for treating superficial tumours, the use of PDT in treating deeply seated cells, such as tumour masses, and anatomically less accessible lesions is limited.

Sonodynamic therapy (SDT) is a more recent concept and involves the combination of ultrasound and a sonosensitising drug (also referred to herein as a “sonosensitiser” or “sonosensitising agent”). In a manner similar to PDT, activation of the sonosensitiser by acoustic energy results in the generation of reactive oxygen species (ROS), such as singlet oxygen, at the target site of interest. Such species are cytotoxic, thereby killing the target cells or at least diminishing their proliferative potential. Many known photosensitising agents can be activated by acoustic energy and are thus suitable for use in SDT. Since ultrasound readily propagates through several cm of tissue, SDT provides a means by which tumours which are located deep within the tissues may be treated. As with light, ultrasound energy can also be focused on a tumour mass in order to activate the sonosensitiser thereby restricting its effects to the target site. SDT offers some significant advantages over PDT: ultrasound is widely accepted as a cost effective and safe clinical imaging modality and, unlike light, can be tightly focused with penetration in soft tissue up to several tens of centimetres depending on the ultrasound frequency used.

In WO 2012/143739, sonosensitisers are conjugated to a gas-filled microbubble to provide a microbubble-sonosensitiser “complex” (or “conjugate”) for use in SDT. These complexes permit effective delivery of the active sonosensitiser in a site-specific manner by a controlled destruction of the bubble using ultrasound. Subsequent or simultaneous sono-activation of the targeted sonosensitiser results in cell destruction at the target site and regression of tumour tissues. The effectiveness of SDT using such complexes for the treatment of pancreatic cancer has been demonstrated in a pre-clinical mouse model bearing human xenograft BxPC-3 tumours (see McEwan et al., J Control Release. 2015; 203, 51-6). In further developments of this work, combined SDT/anti-metabolite therapies have been proposed in WO 2017/089800 and WO 2018/220376. The targeted delivery of a sonosensitiser and an anti-metabolite (e.g. 5-fluorouracil (5-FU) or gemcitabine) using a microbubble—either in the form of a single microbubble carrying both agents or separate microbubbles carrying the different agents—permits effective delivery of both agents to the tumour. Sono-activation of the targeted sonosensitiser results in the generation of ROS which destroy tumour cells at the target site. This action is complimented by the action of the anti-metabolite which exerts its cytotoxic effect directly at the target site. By using a microbubble as a carrier for both agents, non-specific uptake of these by target tissues is reduced. Such therapies thus provide a more targeted approach with improved efficacy and reduced side-effects compared to systemic administration of the anti-metabolite drug alone.

In WO 2017/089800 and WO 2018/220376, microbubbles carrying the therapeutic agents are prepared by attachment of the agents to a pre-formed microbubble via the “avidin-biotin” interaction. A biotinylated microbubble is produced from a combination of different lipids, one of which is biotinylated (lipid-PEG-biotin). The bubble is then incubated with avidin. Once the avidin is bound to the bubble this permits the binding of further biotinylated moieties, such as a biotinylated chemotherapeutic or sonosensitising agent. The linkage between the bubble and selected agents takes the form of a non-covalent “biotin-avidin-biotin” interaction. These earlier methods require a number of synthetic steps to be performed on the bubble after it has been produced. This not only increases the overall complexity and cost of the manufacturing process, but also reduces the yield of the loaded microbubbles. Such methods also provide for a limited degree of control over loading levels of the therapeutic agents onto the bubble.

A need thus exists for alternative (e.g. improved) methods to prepare microbubbles which carry therapeutic agents for use in targeted methods of sonodynamic therapy, in particular combined methods of sonodynamic and anti-cancer therapy. In particular, there is a need for such methods which minimise the need to manipulate the bubble once it has been formed and/or which permit greater control over the therapeutic agent loading levels.

The methods herein disclosed address these needs and provide microbubble-complexes carrying therapeutic agents which are particularly beneficial for use in methods of sonodynamic therapy, for example in methods of combined sonodynamic therapy and chemotherapy. These complexes find particular use in treating deeply-sited cancers, such as pancreatic cancer, which remain difficult to treat using conventional treatment methods.

### SUMMARY OF THE INVENTION

Disclosed herein are methods to produce a microbubble which carries a therapeutic agent, such as a chemotherapeutic agent, a sonosensitising agent, or any combination of such agents, in which at least one of said agents is covalently attached to the shell of the microbubble. The methods involve the formation of a “functionalised lipid” which is covalently bound to at least one therapeutic agent before production of the loaded microbubble (herein referred to as a “microbubble-therapeutic agent complex” or, simply, a “microbubble-complex”). Covalent attachment of the therapeutic agent to the lipid prior to formation of the microbubble avoids the need to manipulate the bubble once it has been produced. The methods of the invention thus provide the advantages, for example, of simplicity and potential cost saving in manufacturing of the microbubble-complex. By avoiding the need for multiple conjugation steps to be carried out on the bubble once it has been formed, the yield of the microbubble-complex is also improved. In addition to this, pre-treatment of the lipid to introduce the selected therapeutic agent(s) allows for greater control over the introduction of the therapeutic agent(s) in the final microbubble-complex—as a result, drug loading levels can be more precisely tailored according to need.

The microbubble-complexes prepared in accordance with the methods herein disclosed also possess a number of surprising, yet beneficial properties.

As evidenced by the results presented herein, for example, the microbubble-complexes of the present disclosure are capable of providing an improved in vivo response at a significantly reduced dose of the selected therapeutic agent(s). This allows for much lower drug concentrations to be used compared to the use of the same drugs in conventional “free” form.

The microbubble-complexes disclosed herein are also believed to be capable of improved delivery of therapeutic agent(s) to target cells or tissues due to the precise nature of the covalent linkage between the agent(s) and the lipids which form the shell structure of the microbubble. For example, where the lipid is a phospholipid, the agent is released to the target cells or tissues in phosphorylated form, which is an active metabolite. This is in contrast to earlier biotin-avidin binding methods in which the agent, once released from the microbubble, requires phosphorylation for activation.

The preparation methods herein described also enable a greater degree of flexibility in the synthetic approach to preparation of the drug-loaded microbubbles. As illustrated in the examples of the present disclosure, for example, the anti-metabolite gemcitabine may be linked via its amine group to the lipid shell of the bubble. Although not wishing to be bound by theory, it is believed that this will reduce its ability to serve as a substrate for cytidine deaminase, thus extending its half-life once delivered in vivo. In certain embodiments, the microbubble-complexes made according to the methods herein disclosed thus have superior characteristics for use in sonodynamic therapy, in particular when used in a combined therapy with chemotherapeutic agents.

As will be discussed herein, the microbubble-therapeutic agent complexes made by the methods of the present disclosure are considered to be novel and thus form a further aspect of the invention.

The microbubble-complexes of the present disclosure are particularly suitable for the treatment of pancreatic cancer using ultrasound. However, their use extends to the treatment of other diseases and conditions, for example those characterised by hyperproliferative and/or abnormal cells, and in particular to the treatment of other deeply-sited cancers and tumours. These therefore have broader application which extends to the treatment of other diseases and conditions which may benefit from treatment by sonodynamic therapy using known therapeutic (e.g. chemotherapeutic) drugs. Other therapeutic agents suitable for use in the invention can readily be determined by those skilled in the art based on the desired treatment method.

In one aspect the invention provides a method of preparing a microbubble covalently attached to at least one therapeutic agent (herein referred to as a “microbubble-therapeutic agent complex” or “microbubble-complex”), said method comprising:
providing a lipid which is capable of forming a microbubble;
covalently linking at least one therapeutic agent to said lipid whereby to produce a functionalised lipid; and
preparing a microbubble from said functionalised lipid.

In another aspect the invention provides a microbubble-therapeutic agent complex obtained or obtainable by any method as herein described.

In another aspect the invention provides a microbubble-therapeutic agent complex which comprises a microbubble shell formed from a plurality of lipids, wherein at least a proportion of said lipids are functionalised lipids which are covalently linked to at least one therapeutic agent.

In another aspect the invention provides a pharmaceutical composition comprising a microbubble-therapeutic agent complex as herein described, together with at least one pharmaceutical carrier or excipient.

In another aspect the invention provides a microbubble-therapeutic agent complex or a pharmaceutical composition comprising a microbbuble-therapeutic agent complex as herein described for use as a medicament or for use in therapy, preferably for use in a method of sonodynamic therapy, e.g. for use in a method of combined sonodynamic therapy and chemotherapy.

In another aspect the invention provides the use of a microbubble-therapeutic agent complex as herein described in the manufacture of a medicament for use in a method of sonodynamic therapy, for example for use in a method of combined sonodynamic therapy and chemotherapy.

In another aspect the invention provides a method of sonodynamic treatment, for example a method of combined sonodynamic and chemotherapeutic treatment, said method comprising the step of administering to a subject in need thereof (e.g. a patient) a pharmaceutically effective amount of a microbubble-therapeutic agent complex, or a pharmaceutical composition containing a microbubble-therapeutic agent complex, as herein defined, and subjecting said complex to ultrasound irradiation whereby to activate said complex.

### DETAILED DESCRIPTION OF THE INVENTION

### Definitions

As used herein, the term “microbubble” is intended to refer to a microsphere comprising a shell having an approximately spherical shape and which surrounds an internal void which comprises a gas or mixture of gases. The “shell” refers to the membrane which surrounds the internal void of the microbubble.

The terms “sonosensitiser”, “sonosensitising agent” and “sonosensitising drug” are used interchangeably herein and are intended to refer to any compound which is capable of converting acoustic energy (e.g. ultrasound) into reactive oxygen species (ROS), such as singlet oxygen, that results in cell toxicity.

As used herein, the terms “sonodynamic therapy” and “sonodynamic treatment” are intended to refer to a method involving the combination of ultrasound and a sonosensitising agent in which activation of the sonosensitising agent by acoustic energy results in the generation of reactive oxygen species, such as singlet oxygen.

As used herein, the term “therapeutic agent” is intended to define any chemical or biological compound that can benefit a human or non-human animal (e.g. a non-human mammal). It encompasses agents capable of the reduction, alleviation or elimination, of a disease, condition or disorder. It includes agents which may have prophylactic effect to minimise, or partially or completely inhibit the development of a disease, condition or disorder.

As used herein, the term “chemotherapeutic agent” is intended to broadly encompass any chemical or biological compound useful in the treatment of cancer. It includes growth inhibitory agents and other cytotoxic agents. The term “growth inhibitory agent” refers to a compound which inhibits growth of a cell, especially a cancer cell.

As used herein, the term “cancer” refers to cells undergoing abnormal proliferation. Growth of such cells typically causes the formation of a tumour. Cancerous cells may be benign, pre-malignant or malignant. Such cells may be invasive and/or have the ability to metastasize to other locations in the body. The term cancer, as used herein, includes cancerous growths, tumours, and their metastases. The term “tumour”, as used herein, refers to an abnormal mass of tissue containing cancerous cells.

As used herein, the term “metastasis” refers to the spread of malignant tumour cells from one organ or part of the body to another non-adjacent organ or part of the body. Cancer cells may break away from a primary tumour, enter the lymphatic and blood systems and circulate to other parts of the body (e.g. to normal tissues). Here they may settle and grow within the normal tissues. When tumour cells metastasize, the new tumours may be referred to as a “secondary” or metastatic cancer or tumour. The term “metastatic disease” as referred to herein relates to any disease associated with metastasis.

As used herein, the term “micrometastasis” refers to a collection of cancer cells (also known as micrometastases or “micromets”) which are shed from a primary tumour and which spread to another part of the body. The term “micrometastatic disease” is used herein in respect of any disease associated with micrometastasis.

The term “circulating tumour cells” (CTCs) refers to cells that are shed into the vasculature or lymphatics from a primary tumour and are carried around the body in the blood. CTCs act as seeds for the subsequent growth of additional tumours (metastases) in other organs or parts of the body.

As used herein, “treatment” includes any therapeutic application that can benefit a human or non-human animal (e.g. a non-human mammal). Both human and veterinary treatments are within the scope of the present invention, although primarily the invention is aimed at the treatment of humans. Treatment is intended to refer to the reduction, alleviation or elimination, of a disease, condition or disorder. It includes palliative treatment, i.e. treatment intended to minimise, or partially or completely inhibit the development of the disease, condition or disorder. Where not explicitly stated, treatment also encompasses prevention. As used herein, “prevention” refers to absolute prevention, i.e. maintenance of normal levels with reference to the extent or appearance of a particular symptom of the disease, condition or disorder, or to reduction or alleviation of the extent or timing (e.g. delaying) of the onset of that symptom.

By “a pharmaceutical composition” is meant a composition in any form suitable to be used for a medical purpose.

As used herein, a “pharmaceutically effective amount” relates to an amount that will lead to the desired pharmacological and/or therapeutic effect, i.e. an amount of the agent which is effective to achieve its intended purpose. While individual subject (e.g. patient) needs may vary, determination of optimal ranges for effective amounts of the active agent(s) herein described is within the capability of one skilled in the art. Generally, the dosage regimen for treating a disease, condition or disorder with any of the therapeutic agents described herein may be selected by those skilled in the art in accordance with a variety of factors, including the nature of the condition and its severity.

The term “pharmaceutically acceptable salt” as used herein refers to any pharmaceutically acceptable organic or inorganic salt of any of the compounds herein described. Examples of suitable pharmaceutically acceptable salts are well known to those of skill in the art.

The term “subject” refers to any individual who is the target of the administration or treatment. The subject may be, for example, a mammal. Thus the subject may be a human or non-human animal. The term “patient” refers to a subject under the treatment of a clinician. Preferably, the subject will be a human.

The term “residue” when used in the context of a “residue” of a particular compound (e.g. a therapeutic agent) refers to the moiety formed when that compound (e.g. a therapeutic agent) has taken part in a reaction to covalently link it to another compound (e.g. to a lipid or suitably modified lipid) as herein described. Covalent linkage may result from reaction of a terminal group of the compound (e.g. therapeutic agent).

As used herein, a “protecting group” refers to a chemical group which can be introduced into a molecule by chemical modification of a functional group to obtain chemoselectivity in a subsequent chemical reaction. Protecting groups may be introduced onto a specific functional group in a polyfunctional molecule to block its reactivity under reaction conditions needed to make modifications elsewhere in the molecule. Suitable protecting groups should be readily, but selectively, introduced into the desired functional group, be stable to the reagents employed in the subsequent reaction steps and, ideally, be capable of being removed under mild conditions when no longer required. Suitable protecting groups are well known to a person skilled in the art.

In one aspect the invention provides a method of preparing a microbubble-therapeutic agent complex. The method involves the step of covalent attachment of a therapeutic agent to a lipid capable of forming a microbubble. By a “lipid capable of forming a microbubble”, it is intended that the lipid should be capable of maintaining a microbubble by forming a layer at the interface between a gas within the core of the microbubble and an external medium, e.g. an aqueous solution which contains the microbubble. As will be understood, a lipid which is “capable of forming a microbubble” is not intended to encompass a lipid which has already been incorporated into the shell structure of a microbubble. Covalent attachment will comprise the formation of at least one covalent linkage between the selected lipid and therapeutic agent, but it may also involve the formation of more than one covalent linkage. Typically, a single lipid will be functionalised by a single covalent linkage to a single therapeutic agent. As will be described herein, covalent linkage of the therapeutic agent and the lipid may be ‘direct’, or it may be ‘indirect’, i.e. via a suitable linking moiety which is covalently attached both to the lipid and to the therapeutic agent.

Lipids for use in the invention may be of natural, semi-synthetic or synthetic origin. As will be understood, the lipids should be biocompatible. Lipids which are suitable for the preparation of a microbubble are known in the art and include, for example, any of the lipids described in WO 2012/143739, WO 2017/089800 and WO 2018/220376, the entire contents of which are incorporated herein by reference.

To produce a microbubble, the lipids for use in the invention may be amphiphilic in character, i.e. having both hydrophilic and hydrophobic properties.

Suitable lipids include, but are not limited to, phospholipids, fatty acids, triglycerides, diglycerides, monoglycerides, sterols and sterol derivatives (e.g. cholesterol), sphingolipids (e.g. sphingomyelin), and combinations thereof.

Lipids containing saturated and/or unsaturated fatty acid groups may be used in the invention. Long chain lipids are generally preferred, for example those containing fatty acid chains having from 10 to 30 carbon atoms, preferably 10 to 25 carbon atoms, e.g. from 12 to 22 carbons in either linear or branched form (preferably in linear form). Any fatty acids herein described may be fluorinated, i.e. these may include one or more (e.g. one) fluorine atom.

Examples of saturated fatty acids that may be present in the lipids for use in the invention include, for example, lauric, myristic, palmitic, stearic, and docosanoic (behenic) acids. Examples of unsaturated fatty acids that may be present include, for example, lauroleic, myristoleic, palmitoleic and oleic acids. Examples of branched fatty acids include, for example, isolauric, isomyristic, isoplamitic and isostearic acids. Saturated fatty acids, especially long chain fatty acids are generally preferred.

Phospholipids are generally preferred for use in the invention. These consist of two hydrophobic fatty acid tail groups and a hydrophilic head containing a phosphate group. The head and tail groups are linked together by a glycerol backbone. The hydrophilic head consists of a phosphate group which may be modified with various organic molecules such as, for example, choline, ethanolamine or serine. The nature of the phospholipid for use in the invention is not particularly limited and includes phospholipids having both saturated and unsaturated fatty acid groups (including fatty acid groups which may be fluorinated). Saturated and unsaturated (including mono- and polyunsaturated) fatty acids include, but are not limited to, molecules having 10 to 30 carbon atoms, preferably 10 to 25 carbon atoms, e.g. 12 to 22 carbon atoms, in either linear or branched form. Examples of fatty acids include any of those listed herein and any of these may optionally be fluorinated.

Non-limiting examples of phospholipids suitable for use in the invention include:
phosphatidylcholines, e.g. dimyristoylphosphatidylcholine, dipalmitoylphosphatidylcholine, distearoylphosphatidylcholine, dioleoylphosphatidylcholine, dipentadecanoylphosphatidylcholine, dilauroylphosphatidylcholine, and 1,2-dibehenoyl-sn-glycero-3-phosphocholine (DBPC);
phosphatidic acids;
phosphatidylethanolamines, e.g. dioleoylphosphatidylethanolamine, and 1,2-distearoyl-sn-glycero-3-phosphoethanolamine (DSPE);
phosphatidylserines;
phosphatidylglycerols, e.g. diphosphatidylglycerols such as cardiolipin; and
phosphatidylinositols.

Suitable phospholipids may be exemplified by the following compounds of formula (I), and pharmaceutically acceptable salts thereof:

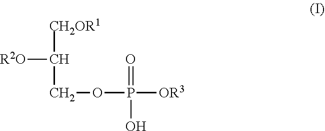

wherein:

R¹ and R², which may be the same or different, are saturated, mono- or polyunsaturated C₁₀₋₃₀ acyl groups, for example —CO—C₁₀₋₂₅ alkyl or —CO—C₁₀₋₂₅ alkenyl groups; and

R³ is

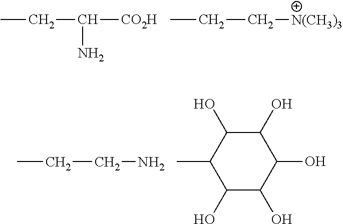

In formula (I), R¹ and R² will typically be derived from saturated fatty acids and are thus —CO-alkyl groups. These may be the same or different, e.g. selected from lauroyl, myristoyl, palmitoyl, stearoyl, oleoyl, linoleoyl, and behenoyl groups.

In one embodiment, the lipids for use in the invention are selected from 1,2-dibehenoyl-sn-glycero-3-phosphocholine (DBPC), 1,2-distearoyl-sn-glycero-3-phosphoethanolamine (DSPE), and combinations thereof. In one embodiment a combination of lipids may be used to prepare the microbubble-complexes in which DBPC is present in an amount of at least 70, preferably at least 80, more preferably at 90 mol. % (based on the total amount of lipid).

Lipids and combinations of different lipids (e.g. combinations of two different lipids) may be used to produce the microbubble-complexes. Where more than one type of lipid is used, it will be understood that at least one of the lipids will be modified as herein described to form a “functionalised lipid” carrying at least one (e.g. one) therapeutic agent via a covalent linkage. However, it is not essential that all lipids used to form the microbubble-complex will be functionalised in this way. Other non-functionalised lipids (i.e. lipids which do not carry any therapeutic agent) may also be used. Such lipids will also be capable of forming a microbubble and may be any of those herein described.

In one embodiment, a proportion of the lipids used to produce the microbubble-complex may be modified to carry one or more biocompatible polymers or polysaccharides, for example a polyalkylene glycol such as polyethylene glycol (PEG). Lipids bearing polymers such as PEG, including but not limited to PEG 2000 MW, PEG 5000 MW, and PEG 8000 MW, are particularly suitable for improving the stability and size distribution of the microbubbles. Different mole ratios of lipids bearing polymers (e.g. a PEGylated lipid) and other lipids may be used. These may be used in combination with one or more functionalised lipids as herein described.

Methods suitable for covalent linkage of the selected lipid and therapeutic agent(s) may readily be determined by those skilled in the art. The choice of method to be used will depend on the chemical structure of the lipid and the therapeutic agent, for example on the nature of any pendant functional groups which may undergo a chemical reaction to form the desired covalent bond. Examples of covalent bonds which may be formed include ester, amide, ether, carbamate, urea, thiourea, sulphide, disulfide, sulfone, and carbonate linkages, and C—C bonds. Where appropriate, the lipid and/or the therapeutic agent may be suitably modified to enable their reaction, for example these may be modified to introduce a suitable functional group.

In some embodiments, the lipid in its conventional (e.g. commercially available) form, whether synthetic, semi-synthetic or natural, may possess a functional group capable of linking to the desired therapeutic agent by a covalent bond. The nature of the functional group present in the lipid is not limited. In the case of a phospholipid, for example, the functional group may be amino in phosphatidylethanolamine, hydroxyl in phosphaditylglycerol, and carboxyl in phosphatidylserine.

In other embodiments, the lipid for use in the invention may be capable of undergoing a transesterification or hydrolysis reaction enabling it to form a covalent link with a hydroxyl-carrying therapeutic agent. As will be understood, such a reaction enables direct linkage of the therapeutic agent to the phosphate ester group of the lipid. The resulting functionalised lipid may, for example, be a compound of formula (II), or a pharmaceutically acceptable salt thereof.

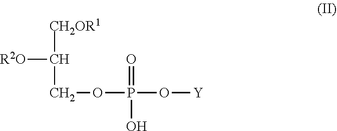

wherein
R¹ and R², which may be the same or different, are as herein defined; and
Y is the “residue” of a therapeutic agent.

In other embodiments, the lipid for use in the invention may be chemically modified prior to reaction with the therapeutic agent. The formation of such a “modified” lipid may be appropriate, for example, where the unmodified lipid is not capable of undergoing a suitable chemical reaction with the chosen therapeutic agent. Modification of the lipid may, for example, alter the functionality of an existing functional group, e.g. the conversion of a carboxylic acid to an amide or ester, etc. Alternatively, the lipid may be modified by reaction with another compound to provide a linking moiety which carries a terminal functional group enabling it to form a covalent link to a therapeutic agent. A non-limiting example of such a modification is the reaction of a lipid with an acid anhydride which provides a linking moiety having a terminal carboxylic acid group. By way of example, a lipid (e.g. a phosphatidylethanolamine) which has been modified in this way may be reacted with a therapeutic agent to provide a “functionalised lipid” of formula (III), or a pharmaceutically acceptable salt thereof:

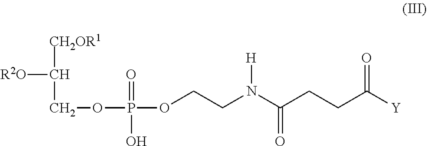

wherein
R¹ and R², which may be the same or different, are as herein defined; and
Y is the “residue” of a therapeutic agent.

The nature of the therapeutic agent for use in the invention is not considered to be limited. Any agent or combination of agents suitable for use in a method of sonodynamic therapy may be used. One or more therapeutic agents may be linked to a single microbubble in accordance with the methods herein described. Where the microbubble is linked to more than one therapeutic agent, these may be the same or different.

The selected therapeutic agent should be capable of covalent linkage to the chosen lipid (or suitably modified lipid as herein described) and may, for example, contain one or more groups capable of forming the desired covalent bond with the lipid. If required, however, the therapeutic agent may be suitably “functionalised”, e.g. to include one or more reactive groups which enable its reaction with the lipid (or modified lipid). Any suitable functional groups may be used and these may readily be selected by those skilled in the art. Suitable functional groups which may be introduced include, for example, carboxylic acid, hydroxyl (e.g. primary hydroxyl), carbonyl, acid halide, thiol and/or amine (e.g. primary amine) groups. Methods for the introduction of such functional groups are well known in the art. Functionalisation may involve reaction of the therapeutic agent with a compound which is capable of providing the desired functionality and may result in the introduction of a linking moiety which enables its reaction with the selected lipid. Suitable compounds may readily be determined by any skilled chemist and include, for example, compounds containing terminal amine or carboxylic acid groups. Following reaction with the agent these may, for example, provide a terminal amine or carboxylic acid functionality which is capable of reaction with the chosen lipid.

Therapeutic agents which comprise a primary hydroxyl group, or which may be modified to introduce such a group, are preferred for use in the invention due to their ease of reaction with a lipid.

Where the lipid or the therapeutic agent are polyfunctional, suitable protecting groups may be used to block the reaction of one or more of the functional groups (e.g. hydroxyl, amine, etc.) to obtain the desired chemoselectivity in the reaction to covalently link the lipid and therapeutic agent. Suitable protecting groups and methods for their use are well known to a person skilled in the art.

The therapeutic agent may, for example, be a chemotherapeutic agent or a sonosensitising agent. Combinations of such agents may also be used to produce the microbubble-complex. For example, in one embodiment the microbubble-complex may comprise a plurality of chemotherapeutic agents which may be the same or different. In another embodiment, it may comprise a plurality of sonosensitising agents which may be the same or different. In another embodiment, the microbubble-complex may comprise a combination of one or more chemotherapeutic agents (which may be the same or different) and one or more sonosensitising agents (which may be the same or different). Whilst it is recognised that certain sonosensitisers may be considered to have chemotherapeutic properties (although their ability to function as a chemotherapeutic may be dependent on their concentration level) and may therefore be considered a “chemotherapeutic agent” within the definition provided herein, it will be understood that where any reference is made herein to any combination of a chemotherapeutic agent and a sonosensitising agent, such agents will not be identical to one another. For example, it is intended that a microbubble which is covalently linked both to a sonosensitising agent and to a chemotherapeutic agent will carry two different types of therapeutic agent.

For use in the invention, suitable classes of chemotherapeutic agents and examples within those classes include any of the following, or suitably functionalised derivatives of such agents: antifolates (e.g. methotrexate); 5-fluoropyrimidines (e.g. 5-fluorouracil or 5-fluorouridine); cytidine analogues (e.g. gemcitabine); purine antimetabolites (e.g. mercaptopurine); alkylating agents (e.g. cyclophosphamide); non-classical alkylating agents (e.g. dacarbazine); platinum analogues (e.g. cisplatin); antitumour antibiotics (e.g. actinomycin D, bleomycin, mitomycin C); bioreductive drugs (e.g. mitomycin C, Banoxantrone (AQ4N)); anthracyclines (e.g. doxorubicin, mitoxantrone); topoisomerase I inhibitors (e.g. irinotecan); topoisomease II inhibitors (e.g. etoposide); antimicrotubule agents such as vinca alkaloids (e.g. vincristine), taxols (e.g. paclitaxel), and epothilones (e.g. ixabepiline); antioestrogens (e.g. tamoxifen); antiandrogens (e.g. biclutamide, cyproterone acetate); aromatase inhibitors (e.g. anastrazole, formestan); antiangiogenic or hypoxia targeting drugs (either naturally occuring, e.g. endostatin, or synthetic, e.g. gefitinib, lenalidomide); antivascular agents (e.g. cambretastatin); tyrosine kinase inhibitors (e.g. gefitinib, erlotinib, vandetanim, sunitinib); oncogene or signalling pathway targeting agents (e.g. tipfarnib, lonafarnib, naltrindole, rampamycin); agents targeting stress proteins (e.g. geldanamycin and analogues thereof); autophagy targeting agents (e.g. chloroquine); proteasome targeting agents (e.g. bortezomib); telomerase inhibitors (targeted oligonucleotides or nucleotides); histone deacetylase inhibitors (e.g. trichostatin A, valproic acid); DNA methyl transferase inhibitors (e.g. decitabine); alkyl sulfonates (e.g. busulfan, improsulfan and piposulfan); aziridines (e.g. benzodopa, carboquone, meturedopa, and uredopa); ethylenimines and methylarmelamines (e.g. altreta.mine, triethylenemelamine, trietylenephosphoramide, triethylenethiophosphaoramide and trimethylolomelamine); nitrogen mustards (e.g. chlorambucil, chlornaphazine, cholophosphamide, estramustine, ifosfamide, mechlorethamine, mechlorethamine oxide hydrochloide, melphalan, novembichin, phenesterine, prednimustine, trofosfamide, uracil mustard); nitrosureas (e.g. carmustine, chlorozotocin, fotemustine, lomustine, nimustine, ranimustine); purine analogues (e.g. fludarabine, 6-mercaptopurine, thiamiprine, thioguanine); pyrinidine analogues (e.g. ancitabine, azacitidine, 6-azauridine, carmofur, cytarabine, dideoxyuridine, doxifluridine, enocitabine, floxuridine); androgens (e.g. calusterone, dromostanolone propionate, epitiostanol, mepitiostane, testolactone); anti-adrenals (e.g. aminoglutethimide, mitotane, trilostane); immune checkpoint inhibitors (e.g. the PD-1/PDL-1 interaction inhibitors BMS-1001 and BMS-1166) and other immune response modifiers (e.g. imiquimod and resiquimod). Pharmaceutically acceptable salts, derivatives or analogues of any of these compounds may also be used.

Examples of grow the inhibitory agents for use in the invention include agents that block cell cycle progression (at a place other than S phase), such as agents that induce G1 arrest and M-phase arrest. Classical M-phase blockers include the vincas (vincristine and vinblastine); taxane family members, including paclitaxel, docetaxel, and analogues thereof; and topoisomerase inhibitors, such as irinotecan, topotecan, camptothecin, lamellarin D, doxorubicin, epirubicin, daunorubicin, etoposide, and bleomycin. Those agents that arrest G1 include, for example, DNA alkylating agents, such as tamoxifen, prednisone, dacarbazine, mechlorethamine, cisplatin, methotrexate, 5-FU, and ara-C.

In one embodiment the chemotherapeutic agent may be an anti-metabolite. Anti-metabolites which are suitable for use in the invention include the 5-fluoropyrimidines, and cytidine analogues. Examples of anti-metabolites which may be used in the invention are 5-fluorouracil (5-FU), 5-fluorouridine (5-FUR) and gemcitabine. 5-FUR and gemcitabine are particularly suitable for use in the preparation methods herein described since these both carry a pendant primary hydroxyl group enabling these to be readily linked to a lipid without further modification, if desired.

Sonosensitising agents which may be used in the invention include compounds which render target cells or tissues hyper-sensitive to ultrasound. In some cases, a sonosensitising agent may be capable of converting acoustic energy (e.g. ultrasound) into ROS that result in cell toxicity. Others may render the target cell or tissues hypersensitive to ultrasound by compromising the integrity of the cell membrane. It is well known that many known sonosensitising agents can facilitate photodynamic activation and can also be used to render cells or tissues hypersensitive to light.

Examples of compounds suitable for use as sonosensitising agents in the invention include phenothiazine dyes (e.g. methylene blue, toluidine blue), Rose Bengal, porphyrins (e.g. Photofrin®), chlorins, benzochlorins, phthalocyanines, napthalocyanines, porphycenes, cyanines (e.g. Merocyanine 540 and indocyanine green), azodipyromethines (e.g. BODIPY and halogenated derivatives thereof), acridine dyes, purpurins, pheophorbides, verdins, psoralens, hematoporphyrins, protoporphyrins and curcumins. Any known analogues or derivatives of these agents may also be used. Suitable derivatives include the pharmaceutically acceptable salts.

Preferred for use as sonosensitisers in the invention are methylene blue, Rose Bengal, indocyanine green (ICG, also known as Cardio Green), and any analogues and derivatives thereof. Structural analogues of the cyanine-based dyes, e.g. structural analogues of ICG and their pharmaceutically acceptable salts may be used, for example the cyanine dyes IR820 and IR783, both of which are commercially available. To improve the ROS generating capability of cyanine dyes it has been proposed to incorporate halogen atoms (e.g. iodine and bromine) into their structure. For example, in US 2013/0231604 (the entire contents of which are incorporated herein by reference) it is proposed that cyanine-based dyes and analogues of such dyes may be modified by incorporation of three iodine atoms on the benzene or napthalene portion of each benzazole or napthazole ring. Any of the polymethine dyes (in particular the cyanines) disclosed in this document may be used as sonosensitising agents in the present invention. Any of the halogenated analogues of the cyanine dyes (e.g. IR783) described in WO 2017/089800 (the entire contents of which are incorporated herein by reference) may also be used in the invention.

Chemical reactions which may be used to covalently link the chosen lipid and therapeutic agent(s) may be determined by those skilled in the art having in mind the nature of the chemical structures of the components to be covalently linked to one another.

Methods for attaching a phospholipid to a compound having a pendant hydroxyl group are, for example, known in the literature. These may involve an enzyme-catalysed reaction between the lipid and therapeutic agent in a biphasic emulsion. For example, Phospholipase D (PLD) is known for use in the hydrolytic conversion of a phospholipid to a phosphatidic acid in the presence of water which can then react with the hydroxyl group of an acceptor. Such transphosphatidylation methods are, for example, described in the following references, the contents of which are incorporated herein by reference: Shuto et al., Chem, Pharm, Bull, 35(1): 447-449, 1987; Shuto et al., Tetrahedron Letters 28(2): 199-202, 1987; Shuto et al., Nucleosides & Nucleotides 11(2-4): 437-446, 1992; Shuto et al., Bioorganic & Medicinal Chemistry 3(3): 235-243, 1995; Shuto et al., Bioorganic & Medicinal Chemistry Letters 6(9): 1021-1024, 1996; and Hirche et al., Enzyme and Microbial Technology 20: 453-461, 1987). The PLD is not subject to any limitations, although that derived from Streptomyces, for example from Streptomyces chromofuscus, from cabbage, or from Arachis hypogaea (peanut), is considered particularly suitable.

Appropriate reaction conditions for enzyme-catalysed transphosphatidylation may readily be determined by those skilled in the art. Typically, the selected lipid, therapeutic agent and enzyme are provided in a two phase system consisting of a suitably buffered aqueous phase and an organic phase. The enzymatic reaction takes place at the phase boundary of the aqueous and organic phase. Suitable divalent metal ions may be required for enzymatic activity dependent on the selected enzyme. If required, these ions may be provided by calcium salts, such as calcium chloride, which are provided in the aqueous buffer. The pH of the aqueous phase will generally range from 3 to 7, preferably from 4 to 6, e.g. it may be about 4.5. The temperature of the reaction may range from about 25° C. to about 50° C., preferably from about 40° C. to about 50° C., e.g. about 45° C. A variety of organic solvents can be used depending on the solubility of the lipid such as, but not limited to, diethyl ether, ethyl acetate, benzene, and hexane.

A non-limiting example of a PLD-catalysed method for covalent attachment of gemcitabine to the lipid DBPC is shown in the following reaction scheme:

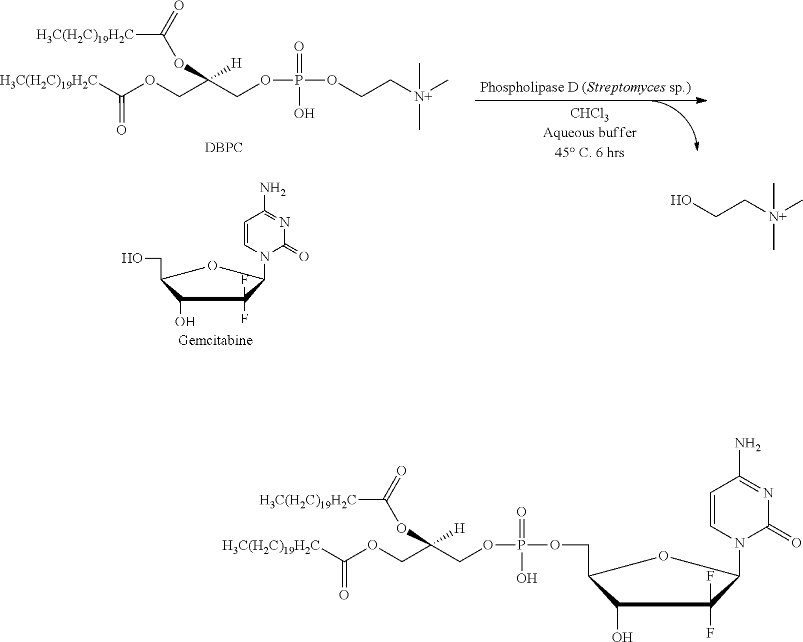

Other non-enzymatic methods may also be employed to covalently link the therapeutic agent to a lipid (e.g. a phospholipid) to provide the desired “functionalised lipid”. In one embodiment of such methods, the lipid may be reacted with another compound (e.g. an acid anhydride) to provide a modified lipid prior to covalent linkage to the selected therapeutic agent. For example, where the lipid is a phosphatidylethanolamine, the method for preparing the “functionalised lipid” may be illustrated by way of the following general reaction schemes to produce a “functionalised lipid” of formula (IV) or (V), or a pharmaceutically acceptable salt thereof:

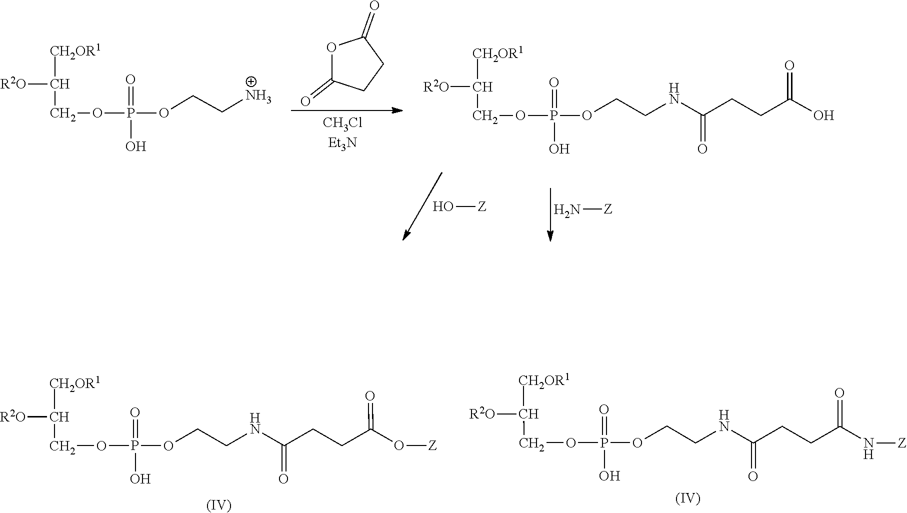

wherein R¹ and R², which may be the same or different, are as herein defined; and
Z is the “residue” of a therapeutic agent.

In these reactions, the therapeutic agent (HO—Z or H₂N—Z) may be employed in the form of a suitably protected derivative thereof in order to direct its point of covalent linkage to the modified lipid. Where any protecting groups are employed, it will be understood that the final step of the process to produce the functionalised lipids will typically involve the removal of the protecting group(s).

Any of the intermediates formed in any of the methods herein described are also considered to form part of the invention, for example any of the intermediates produced in the procedures shown in Schemes 1 to 4 in the accompanying Examples. In particular, any of the functionalised lipids either generally or specifically described herein, including “Lipid-Gem” and “Lipid-5FUR” produced in Examples 1 and 5, and the gemcitabine-functionalised lipids produced in Examples 6 and 7, are considered to form part of the invention, as are any of the methods used for their preparation.

Methods for the formation of microbubbles comprising lipid shell materials are generally known in the art and any known method may be used to convert the functionalised lipids herein described into the desired microbubble-complex. Typically such methods will include dispersion of the selected gas in an aqueous suspension which contains the functionalised lipid(s). Techniques which may be used to form the microbubble from this suspension include sonication, mechanical agitation (e.g. high speed mixing), coaxial electrohydrodynamic atomisation and microfluidic processing using a T-junction (see e.g. Stride & Edirisinghe, Med. Biol. Eng. Comput., 47: 883-892, 2009). Mechanical agitation (e.g. high speed mixing) and sonication techniques are generally preferred, in particular high speed mixing methods.

For example, an aqueous suspension comprising the functionalised lipids and containing one or more stabilising agents may provide a suitable particulate suspension. Other non-functionalised lipids, or other lipids bearing polymer groups as herein described, may also be present. Examples of stabilising agents include, but are not limited to, glycerol, cetyl alcohol, sorbitol, polyvinylalcohol, polypropylene glycol, and propylene glycol. In one embodiment a mixture of glycerol and propylene glycol may be used. Solvent systems suitable for the suspension of the lipids may readily be selected. A preferred solvent system may include saline (e.g. phosphate buffered saline), glycerol and propylene glycol, for example in a ratio of 8:1:1.

Agitation of the resulting suspension of lipids in the presence of the selected gas produces a stable suspension of gas-filled microbubbles which, if desired, can then be separated from the solution. Agitation of the suspension must involve sufficient force that the gas is introduced into the aqueous solution and to allow the formation of the microbubbles. Typically, agitation will involve high speed mixing or sonication.

Sonication may be carried out using an ultrasound transmitting probe. For example, the aqueous suspension of the lipid(s) may be sonicated in the presence of the relevant microbubble component gas to produce the microbubble-complex. Sonication methods known in the art which may be used in preparation of the microbubbles include, but are not limited to, those described in WO 2012/143739, WO 2017/089800 and WO 2018/220376, the entire contents of which are incorporated herein by reference.

Where sonication is employed, the required duration of sonication may be determined by detection of the formation of the gas-filled microbubbles, for example the formation of a milky-white suspension. In one embodiment, more than one sonication cycle may be performed. For example, two sonication cycles may be carried out. The first cycle may involve sonicating the suspension to fully disperse the lipids and will generally be carried out at low power (e.g. amplitude setting about 20%) with the probe tip of the sonicator fully submerged in the liquid for about 30 seconds. The second cycle may involve sonicating the lipid suspension at a higher power (e.g. amplitude setting about 90%) with the probe tip at the gas-liquid interface and under a headspace of the selected gas (e.g. PFB) for about 30 seconds. The frequency of the probe sonicator may suitably be set at about 20 KHz.

Mechanical agitation, for example by high speed mixing, of the lipid-containing suspension may also be employed to produce the desired gas-filled microbubbles. Suitable shaking frequencies and duration may readily be selected by those skilled in the art. The formation of a milky-white suspension may be taken as an indication of the formation of the desired gas-filled microbubbles. A shaking frequency of about 4530±100 oscillations per minute and/or a shaking duration of about 45 seconds may, for example, be employed.

The concentration of lipid required to form the microbubbles will vary depending on the type of lipid used, but may readily be determined by those skilled in the art. For example, in preferred embodiments, the concentration of lipid used to form the gas-filled microbubbles may be about 2.0 mmol/L, e.g. about 2.2 mmol/L, based on the amount of saline solution.

In one embodiment, the functionalised lipids may be dissolved in an organic solvent which is then evaporated to produce a dried lipid film prior to their conversion to the microbubble-complex. The dried lipid film may be reconstituted in a suitable solvent prior to agitation (e.g. sonication or high speed mixing) to produce the loaded microbubbles. Reconstitution of the dried lipids in a suitable aqueous solvent prior to mixing with the gas ensures that the lipids are introduced into an aqueous solution. The step of reconstitution may involve heating of the aqueous solution above the lipid transition temperature with gentle stirring.

For storage prior to use, the loaded microbubbles may be suspended in an aqueous solution, such as a saline (e.g. phosphate buffered saline) solution.

In an alternative method to prepare the microbubbles, an aqueous suspension comprising the functionalised lipids may be lyophilised, for example using a suitable cryoprotectant. The resulting powder can then be reconstituted in a suitable aqueous medium in the presence of the selected microbubble component gas. Reconstitution may, for example, be carried out at the point of use. Such methods involving the formation of a lyophilised powder include those described in U.S. Pat. No. 5,686,060, the contents of which are incorporated herein by reference.

The microbubble-complex produced according to the methods herein described will comprise a lipid shell which is covalently linked to the therapeutic agent(s) and which surrounds an internal void comprising a gas. To the extent that such a complex is intended for use in methods of SDT, it will be ultrasound-responsive. Specifically, it is intended that the microbubble component of the complex can be ruptured by the application of ultrasound, thereby releasing the therapeutic agent(s) at the desired target site. As herein described, activation of any sonosensitising agent(s) present in the complex by acoustic energy will also result in the generation of reactive oxygen species, such as singlet oxygen, which are cytotoxic.

Generally, the microbubbles herein described will be approximately spherical in shape. The size of the microbubble should be such as to permit its passage through systemic circulation (e.g. the pulmonary system) following administration, e.g. by intravenous injection. The microbubbles will typically have a diameter of less than about 200 μm, preferably in the range from about 0.05 to about 100 μm, more preferably about 0.1 to about 100 μm, e.g. from about 0.5 to about 100 μm. Particularly suitable are microbubbles having a diameter of less than about 10 μm, more preferably 1 to 8 μm, particularly preferably up to 5 μm, e.g. 1 to 3 m or about 2 μm. The lipid shell of the microbubble may vary in thickness depending on the choice of lipid(s) used to prepare it and will typically range from about 5 to about 200 nm, e.g. from about 10 to about 200 nm. The precise thickness is not critical provided that the lipid shell performs the desired function of retaining the gas core.

The microbubble shells may comprise single or multiple layers of the same or different lipids. Multiple layers may, for example, be formed in cases where a proportion of the lipids carry one or more polymers or polysaccharides, such as polyethylene glycol (PEG) and polyvinylpyrrolidone.

The gas to be provided within the core of the microbubble should be biocompatible. The term “gas” encompasses not only substances which are gaseous at ambient temperature and pressure, but also those which are in liquid form under these conditions. Where the “gas” is liquid at ambient temperature this will generally undergo a phase change to a gas or vapour at a temperature of 38° C. or above. For any gas which is a liquid at ambient temperature, it is generally preferred that this will undergo a phase change to a gas at a temperature between about 38 and 45° C., preferably slightly above body temperature. For example, it may undergo a phase change when subjected to a stimulus, such as ultrasound, which causes a local increase in temperature. Any reference herein to “gas” should thus be considered to encompass not only gases and liquids, but also liquid vapours and any combination thereof, e.g. a mixture of a liquid vapour in a gas.

Gases which are suitable for incorporation within the microbubbles include air, nitrogen, oxygen, carbon dioxide, hydrogen; inert gases such as helium, argon, xenon or krypton; sulphur fluorides such as sulphur hexafluoride, disulphur decafluoride; low molecular weight hydrocarbons such as alkanes (e.g. methane, ethane, propane, butane), cycloalkanes (e.g. cyclopropane, cyclobutane, cyclopentane), alkenes (e.g. ethylene, propene); and alkynes (e.g. acetylene or propyne); ethers; esters; halogenated low molecular weight hydrocarbons; and mixtures thereof. Examples of suitable halogenated hydrocarbons are those which contain one or more fluorine atoms and include, for example, bromochlorodifluoromethane, chlorodifluoromethane, dichlorodifluoromethane, bromotrifluoromethane, chlorotrifluoromethane, chloropentafluoroethane, dichlorotetrafluoroethane, chlorotrifluoroethylene, fluoroethylene, ethyl fluoride, 1,1-difluoroethane and perfluorocarbons. Examples of suitable fluorocarbon compounds include perfluorocarbons. Perfluorocarbons include perfluoroalkanes such as perfluoromethane, perfluoroethane, perfluoropropanes, perfluorobutanes, perfluoropentanes, perfluorohexanes and perfluoroheptanes; perfluoroalkenes such as perfluoropropene, perfluorobutenes; and perfluorocycloalkanes such as perfluorocyclobutane. Microbubbles containing perfluorinated gases, in particular, perfluorocarbons such as perfluoropropanes, perfluorobutanes, perfluoropentanes and perfluorohexanes are suitable for use in the invention due to their stability in the bloodstream.

In one embodiment, the microbubbles may carry oxygen (e.g. oxygen gas). As oxygen is a key substrate for SDT and many cancers are hypoxic, filling the core of the bubble with oxygen gas enhances the sonodynamic effect and the amount of singlet oxygen produced.

In certain embodiments, the methods herein described may be modified to provide a microbubble-complex which additionally incorporates one or more hydrophobic agents, e.g. a hydrophobic chemotherapeutic agent, within its shell structure. For example, the resulting microbubble-complex may comprise a shell having incorporated therein one or more chemotherapeutic agents. Where the microbubble is also covalently linked to a chemotherapeutic agent (or agents), the chemotherapeutic in the shell of the bubble may be the same or different to any chemotherapeutic agent which is covalently linked to it. In one embodiment, they will be different agents. By further modifying the microbubble-complex to incorporate a chemotherapeutic drug within its shell structure, a highly targeted anti-cancer therapy can be provided having enhanced therapeutic effects.

In one embodiment, the methods herein described may thus further comprise the step of loading a hydrophobic chemotherapeutic agent into the hydrophobic tail region of one or more of the lipids which is to be used to produce the microbubble-complex. Typically, these will be loaded into the hydrophobic tail of a lipid bearing a polymer as herein described, such as PEG.

For incorporation within the shell structure of the resultant microbubble-complex, any of the chemotherapeutic agents herein described may be chosen. Preferably, the chemotherapeutic agent should be one capable of spontaneously embedding within the hydrophobic lipid chains of the selected lipids. This may involve direct hydrophobic interaction in cases where the chemotherapeutic is hydrophobic. In one embodiment, the chemotherapeutic agent for incorporation within the shell of the microbubble may therefore be hydrophobic. Hydrophobic agents may be considered to be those having a Log P value greater than about 2. Alternatively, non-polar chemotherapeutic agents may be suitably modified (e.g. functionalised) by the introduction or one or more non-polar functional groups which enable them to spontaneously embed within the lipid shell of the microbubble-complex.

In one embodiment, the chemotherapeutic agent to be incorporated within the shell of the microbubble-complex may be an anti-microtubule agent. Examples of such agents include, in particular, taxols such as paclitaxel. Paclitaxel (or “PTX”) is hydrophobic. In another embodiment, an immune checkpoint inhibitor (e.g. BMS-1001 or BMS-1166) may be included within the shell of the microbubble-complex.

In the case where a chemotherapeutic agent (e.g. paclitaxel, PTX) is to be incorporated within the shell of the microbubble-complex this may, for example, be dissolved in an organic solvent and added to a solution containing the lipids prior to formation of the microbubble-complex as described herein.

The choice of any of the chemotherapeutic agents for use in the invention will be dependent on various factors such as its intended use, e.g. the nature of the tumour, the patient to be treated, etc., but can readily be selected by those skilled in the art according to need.

The microbubble-complex produced by any of the methods herein described will comprise a microbubble covalently linked to one or more therapeutic agents. In certain embodiments, it may also comprise a chemotherapeutic agent embedded in the shell structure of the bubble. Such microbubble-complexes are considered to be novel and to form a further aspect of the invention. It will be understood that any of the present disclosure relating to the nature of the lipids, the selected therapeutic agents, any linking agents which may be used to covalently link these, the resulting functionalised lipids, etc., also extends to the microbubble-complexes according to the invention.

Depending on the nature of the microbubble-complex and, in particular, the nature of the therapeutic agents which form part of the complex, this may be used without further modification in a method of sonodynamic therapy, or in a method of combined sonodynamic and chemotherapy, or it may be further modified prior to use. For example, the microbubble-complex may comprise one or more chemotherapeutic agents, in addition to one or more sonosensitising agents, i.e. it acts as a carrier for both agents and is able to simultaneously deliver both to the target site. In other embodiments, the complex may comprise only one or more sonosensitising agents. Any complex covalently linked to a sonosensitising agent may, if desired, be used without further modification in a method of sonodynamic therapy, such as any of the methods which are described in WO 2012/143739, WO 2017/089800 and WO 2018/220376, the entire contents of which are incorporated herein by reference.

Where the microbubble-complex carries both a sonosensitising agent and a chemotherapeutic agent (which may be covalently linked to the microbubble and/or incorporated in its shell structure) it may be used without the need for any further modification in a method of combined sonodynamic and chemotherapy.

Where the microbubble-complex carries only a sonosensitising agent (or sonosensitising agents), this may be used alone in a method of sonodynamic therapy. However, to improve the efficacy of the treatment, it can also be used in combination with a separate microbubble-chemotherapeutic complex. The microbubble-chemotherapeutic agent complex may be any known complex in which a microbubble is linked to or otherwise associated with one or more chemotherapeutic agents, for example any of the complexes known and described in WO 2012/143739, WO 2017/089800 and WO 2018/220376. Alternatively, the microbubble-chemotherapeutic complex may be one produced according to any of the methods herein described in which the bubble is covalently linked to one or more chemotherapeutic agents and which, optionally, may also carry the same or a different chemotherapeutic agent embedded in its shell. The use of a microbubble (or microbubbles) to simultaneously deliver both a sonosensitiser and a chemotherapeutic agent confers a number of advantages when used in methods of sonodynamic therapy. Specifically, the delivery of both a sonosensitiser and a chemotherapeutic agent in the form of a complex with a microbubble permits effective delivery of both agents in a site-specific manner (e.g. to an internal tumour) by a controlled destruction of the bubbles using ultrasound. Sono-activation of the targeted sonosensitiser results in the generation of ROS which destroy tumour cells at the target site. This action is complimented by the action of the chemotherapeutic agent (e.g. an anti-metabolite) which exerts its cytotoxic effect directly at the intended target site. By using a microbubble as a carrier for both agents, non-specific uptake of these by non-target tissues is reduced, thus providing a significant advantage over systemic delivery. This therapy is thus expected to reduce side-effects and, in turn, provide significant patient benefit.

Where the microbubble-complex produced according to any of the methods herein described is not covalently linked to any sonosensitising agent, for example it comprises only a chemotherapeutic agent or agents (covalently linked to the bubble and, optionally, also incorporated in its lipid shell), this can be employed in a method of sonodynamic therapy in combination with a separate microbubble-sonosensitiser complex. The separate microbubble-sonosensitiser complex may be any known agent in which a microbubble is linked to or otherwise associated with one or more sonosensitisers, for example any of the complexes known and described in WO 2012/143739, WO 2017/089800 and WO 2018/220376. Alternatively, the microbubble-sonosensitiser may be one produced according to any of the methods herein described in which the bubble is covalently linked to one or more sonosensitising agents.

Where appropriate or desirable, any of the microbubble-complexes produced according to the methods herein described may be further modified by methods known in the art for the introduction of a sonosensitising agent and/or a chemotherapeutic agent onto a pre-formed microbubble. Such methods may involve, for example, linkage of any such agents to the microbubble via the biotin-avidin-biotin interaction such as described, for example, in any of WO 2012/143739, WO 2017/089800 and WO 2018/220376. Such methods typically involve biotinylation of the selected chemotherapeutic or sonosensitising agent and linkage of this to a suitably avidin-functionalised microbubble. Such agents may be linked directly to the microbubble or they may be linked via one or more linking groups. For example, they may be attached to the microbubble via a common linking group which carries more than one agent. Suitable linking groups, such as a tripodal linking group (which comprises three branches), include those described in WO 2018/220376, the entire content of which is incorporated herein by reference. In this embodiment, a first branch of the linking group may be be capable of binding to the microbubble (e.g. via a non-covalent interaction such as “avidin-biotin”), a second branch may be capable of linking to a chemotherapeutic agent (e.g. covalently) and a third branch may be capable of linking to a sonosonsensiting agent (e.g. covalently).

The microbubble-therapeutic agent complexes herein described are suitable for the treatment of any disorders or abnormalities of cells or tissues within the body which are responsive to sonodynamic therapy. These include malignant and pre-malignant cancer conditions, such as cancerous growths or tumours, and their metastases; tumours such as sarcomas and carcinomas, in particular solid tumours. The invention is particularly suitable for the treatment of tumours, especially those which are located below the surface of the skin.

Non-limiting examples of tumours that may be treated using the methods herein described are sarcomas, including osteogenic and soft tissue sarcomas; carcinomas, e.g. breast, lung, cerebral, bladder, thyroid, prostate, colon, rectum, pancreas, stomach, liver, uterine, hepatic, renal, prostate, cervical and ovarian carcinomas; lymphomas, including Hodgkin and non-Hodgkin lymphomas; neuroblastoma, melanoma, myeloma, Wilm's tumour; leukemias, including acute lymphoblastic leukaemia and acute myeloblastic leukaemia; astrocytomas, gliomas and retinoblastomas. In particular, the following tumours and any associated metastatic condition may be treated: pancreatic cancer, breast cancer, prostate cancer, glioma, non-small cell lung carcinoma, head and neck cancers, cancers of the urinary tract, kidney or bladder, advanced melanoma, oesophageal cancer, colon cancer, hepatic cancer, and lymphoma. Metastatic disease, micrometastatic disease or CTCs arising from any of these tumours may also be treated using any of the methods herein described. The treatment of pancreatic cancer forms a preferred aspect of the invention.

In another aspect, the invention thus provides a microbubble-therapeutic agent complex as herein described for use in a method of sonodynamic treatment, for example for use in a combined method of sonodynamic and chemotherapeutic treatment. Corresponding methods of medical treatment also form an aspect of the invention.

For use in any of the methods of sonodynamic therapy herein described, the microbubble-complex (or complexes, where different types of microbubble-complex are intended to be used in combination) will generally be provided in a pharmaceutical composition together with at least one pharmaceutically acceptable carrier or excipient. Such pharmaceutical compositions form a further aspect of the invention. Where different microbubbles are used to carry the chemotherapeutic agent and the sonosensitiser it is envisaged these will generally be co-administered in a single pharmaceutical preparation, e.g. an aqueous solution. However, in another embodiment these may be administered separately (e.g. either simultaneously or sequentially) in separate formulations.

Suitable pharmaceutical compositions may be formulated using techniques well known in the art. Their route of administration will depend on the intended use. Typically, these will be administered systemically and may thus be provided in a form adapted for parenteral administration, e.g. by intradermal, subcutaneous, intraperitoneal or intravenous injection. Suitable pharmaceutical forms thus include, but are not limited to, suspensions and solutions which contain the microbubble complex (or complexes) together with one or more inert carriers or excipients. Suitable carriers include saline, sterile water, phosphate buffered saline and mixtures thereof. The compositions may additionally include other agents such as emulsifiers, suspending agents, dispersing agents, solubilisers, stabilisers, buffering agents, wetting agents, preserving agents, etc. The compositions may be sterilised by conventional sterilisation techniques. Solutions containing the complex (or complexes) may be stabilised, for example by the addition of agents such as viscosity modifiers, emulsifiers, solubilising agents, etc.

Typically, the pharmaceutical compositions will be used in the form of an aqueous suspension of the microbubble-complex (or complexes) in water or a saline solution, e.g. phosphate-buffered saline. The complex (or complexes) may be supplied in the form of a lyophilised powder for reconstitution at the point of use, e.g. for reconstitution in water, saline or PBS.

The methods herein described involve administration of a pharmaceutically effective amount of the composition which contains the microbubble-complex (or complexes). The microbubble-complex (or complexes) may then be allowed to distribute to the desired portion or target area of the body prior to activation. Once administered to the body, the target area is exposed to ultrasound at a frequency and intensity to achieve the desired therapeutic effect. A typical activation procedure may involve a two-step process in which the microbubbles are first ruptured by ultrasound thereby releasing the sonosensitiser and/or the chemotherapeutic agent which is then able to penetrate the desired target tissue (e.g. tumour). Subsequent sono-activation of the sonosensitiser in or around the target cells results in production of singlet oxygen which can oxidise various cell components such as proteins, lipids, amino acids, nucleic acids, and nucleotides/nucleosides thereby destroying the target cells. Whilst it is envisaged that activation of the sonosensitiser will typically take place subsequent to its delivery (i.e. following burst of the microbubbles to release the sonosensitiser), delivery of the complex and activation of the sonosensitiser may nevertheless be simultaneous.

Alternatively, any of the methods herein described may involve exposure of the target area in the body to ultrasound during administration of the composition which contains the microbubble-complex (or complexes), i.e. administration of the microbubble-complex (or complexes) and delivery of ultrasound may be carried out simultaneously. Where the half-life of the microbubble-complex is low, this can avoid the situation in which a significant proportion may be removed before the target area receives the ultrasound.

The effective dose of any of the compositions herein described will depend on the nature of the microbubble-complex, the mode of administration, the condition to be treated, the patient, etc. and may be adjusted accordingly.

The frequency and intensity of the ultrasound which may be used can be selected based on the need to achieve selective destruction of the microbubble at the target site and may, for example, be matched to the resonant frequency of the microbubble. Ultrasound frequencies will typically be in the range 20 kHz to 10 MHz, preferably 0.1 to 2 MHz. Ultrasound may be delivered as either a single frequency or a combination of different frequencies. Intensity (i.e. power density) of the ultrasound may range from about 0.1 W/cm² to about 1 kW/cm², preferably from about 1 to about 50 W/cm². Treatment times will typically be in the range of 1 ms to 20 minutes and this will be dependent on the intensity chosen, i.e. for a low ultrasound intensity the treatment time will be prolonged and for a higher ultrasound intensity the treatment time will be lower. Ultrasound may be applied in continuous or pulsed mode and may be either focused or delivered as a columnar beam.

Any radiation source capable of producing acoustic energy (e.g. ultrasound) may be used in the methods herein described. The source should be capable of directing the energy to the target site and may include, for example, a probe or device capable of directing energy to the target tissue from the surface of the body.

In cases where the ultrasound frequencies and/or intensities that are needed to achieve cavitation (or microbubble destruction) and those required to cause sonosensitiser activation are different, these different sets of ultrasound parameters (frequency/intensity) may be applied simultaneously or in a two (or multiple)-step procedure. In the case where the sonosensitiser used is one which also responds to light, ultrasound activation may be accompanied by light activation.

The invention will now be described further with reference to the following non-limiting Examples and the accompanying figures in which:

FIG. 1 is a matrix-assisted laser desorption/ionization-time of flight (MALDI-TOF) spectrum of “Lipid-Gem” (m/z 500-2000).

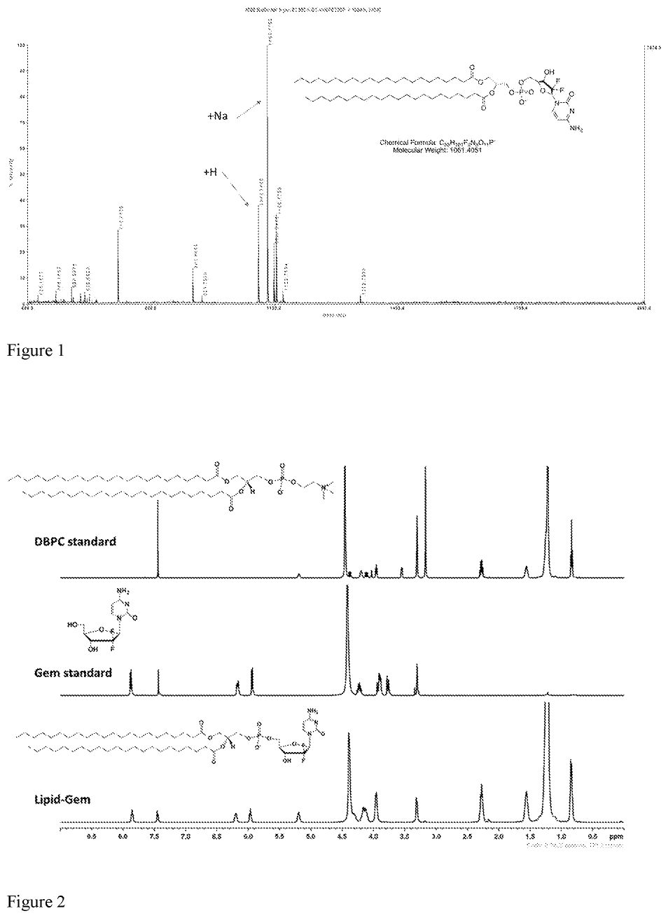

FIG. 2 shows stacked ¹H NMR spectra of 1,2-dibehenoyl-sn-glycero-3-phosphocholine standard (top), gemcitabine standard (middle) and “Lipid-Gem” (bottom) recorded in a CDCl₃/MeOD mixed solvent system.

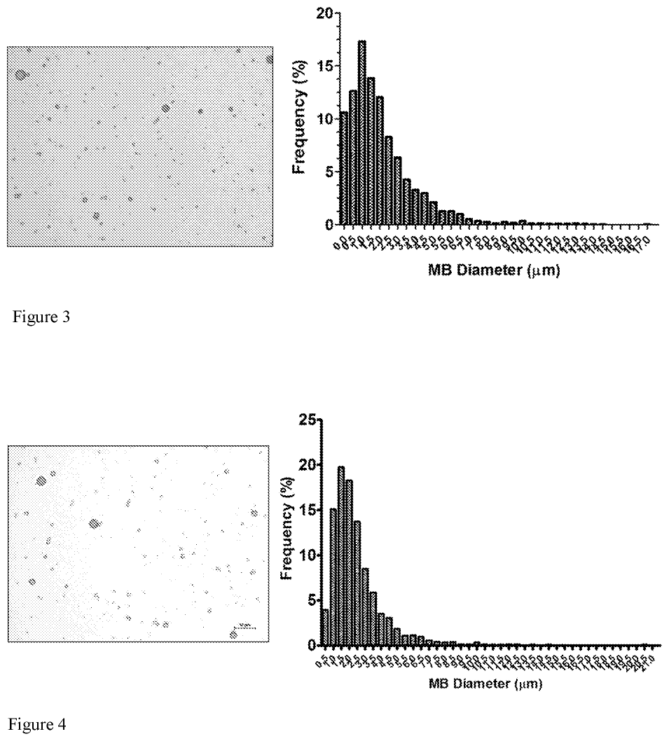

FIG. 3 shows (a) a representative optical micrograph of “Lipid-Gem” microbubbles (1:25 dilution, ×40 magnification); and (b) representative size distribution analysis of images of “Lipid-Gem” microbubbles.

FIG. 4 shows (a) a representative optical micrograph of “Lipid-Gem+DSPE-PEG (2000)+PTX” microbubbles (1:25 dilution, ×40 magnification); and (b) representative size distribution analysis of images of “Lipid-Gem+DSPE-PEG (2000)+PTX” microbubbles.

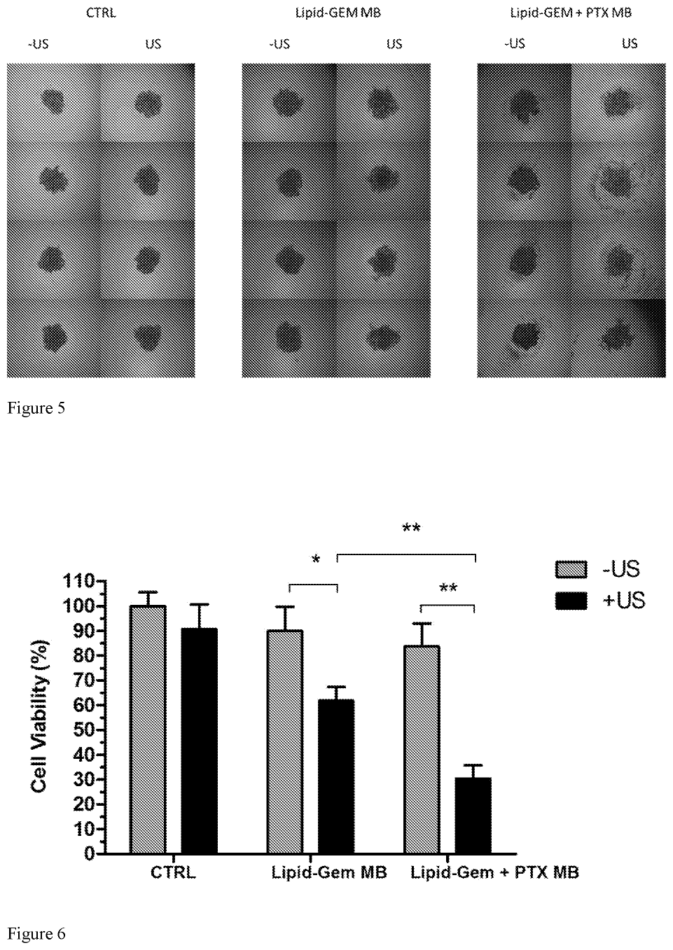

FIG. 5 shows representative images of human pancreas ductal adenocarcinoma cell line (Panc-1) spheroids treated with: (a) no treatment +(left) and −(right) ultrasound; (b) “Lipid-Gem” microbubbles ([Lipid-Gem]=10 μM) +(left) and −(right) ultrasound; and (c) “Lipid-Gem+PTX” microbubbles ([Lipid-Gem]=10 μM, [PTX]=6.2 μM)+(left) and −(right) ultrasound.

FIG. 6 is a plot of cell viability of the Panc-1 spheroids shown in FIG. 5 treated with: (a) no treatment; (b) “Lipid-Gem” microbubbles ([Lipid-Gem]=10 μM); and (c) “Lipid-Gem+PTX” microbubbles ([Lipid-Gem]=10 μM, [PTX]=6.2 μM) in the absence (grey bar) and presence (black bar) of ultrasound.

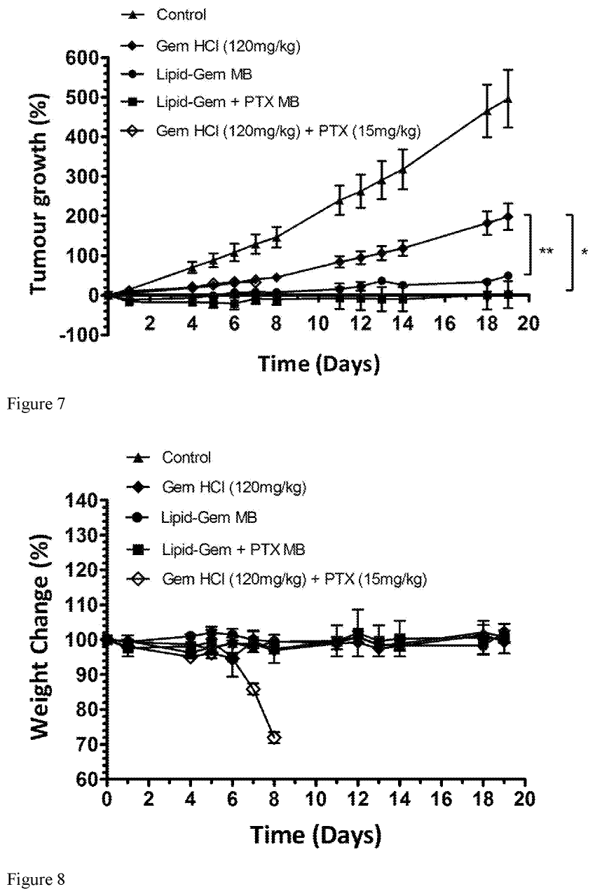

FIG. 7 is a plot of tumour growth in mice bearing BxPC-3 tumours that were either: (i) untreated (upward triangles); or treated with (ii) 100 μL I.V injection of “Lipid-Gem” microbubble suspension plus ultrasound (circles); (iii) 100 μL I.V injection of “Lipid-Gem+PTX” microbubble suspension plus ultrasound (squares); (iv) 100 μL I.P injection of Gem HCl (120 mg/kg) (filled diamonds); or (v) 100 μL of IP injection of Gem HCl (120 mg/kg)+Free PTX (15 mg/kg) (hollow diamonds).

FIG. 8 is a plot of % change in body weight for animals that were either: (i) untreated (triangles); or treated with (ii) 100 μL I.V injection of “Lipid-Gem” microbubble suspension plus ultrasound (circles); (iii) 100 μL I.V injection of “Lipid-Gem+PTX” microbubble suspension plus ultrasound (squares); (iv) 100 μL I.P injection of Gem HCl (120 mg/kg) (filled diamonds); or (v) 100 μL of I.P injection of Gem HCl (120 mg/kg)+Free PTX (15 mg/kg) (hollow diamonds).

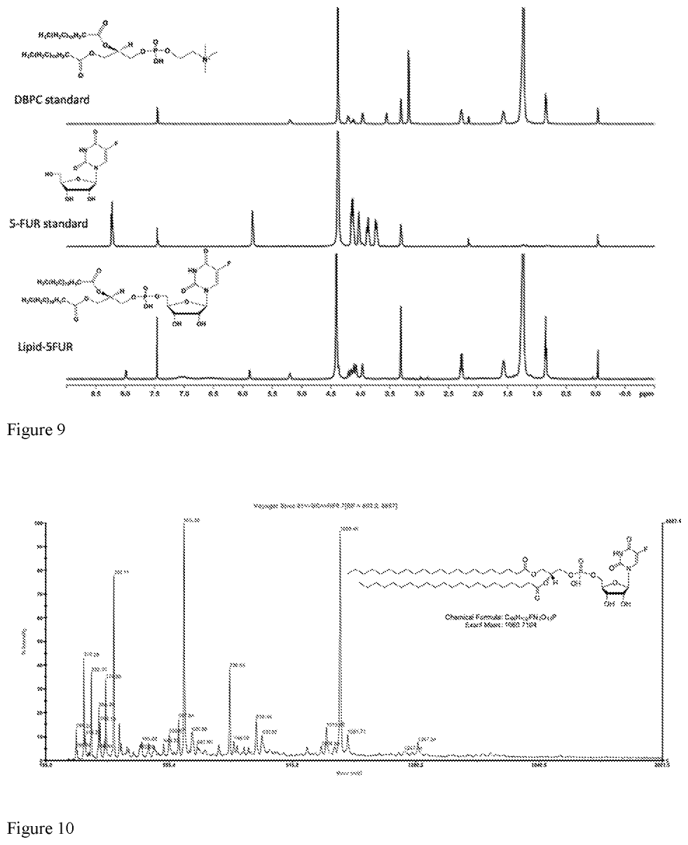

FIG. 9 shows stacked ¹H NMR spectra of 1,2-dibehenoyl-sn-glycero-3-phosphocholine standard (top), 5-flourouridine standard (middle) and “Lipid-5FUR” (bottom).

FIG. 10 is a matrix-assisted laser desorption/ionization-time of flight (MALDI-TOF) spectra of “Lipid-5FUR” (m/z 199-2000) in negative mode.

FIG. 11 is a matrix-assisted laser desorption/ionization-time of flight (MALDI-TOF) spectra of “Lipid-Gem amide (18:0)” (m/z 199-2000) in negative mode. The base peak at 1090.33 corresponds to the [M−H]⁻ ion.

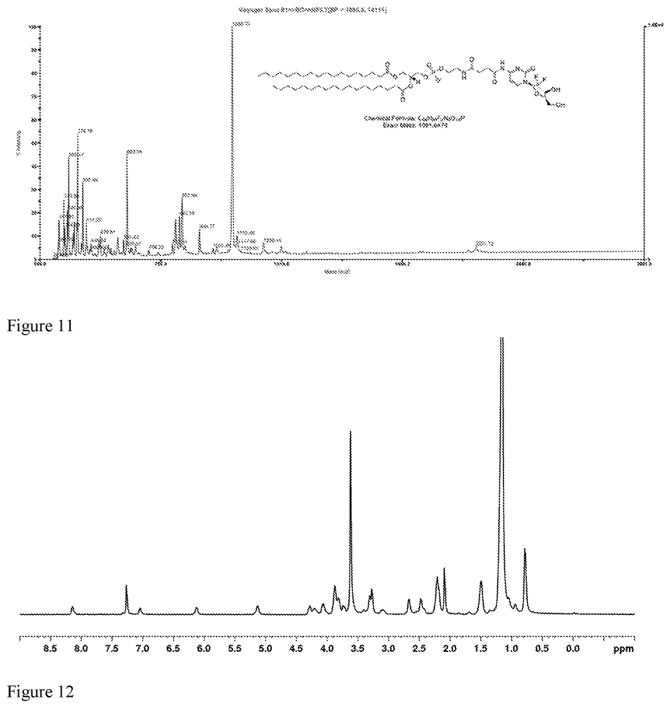

FIG. 12 shows the ¹H NMR spectra of “Lipid-Gem amide (18:0)”.

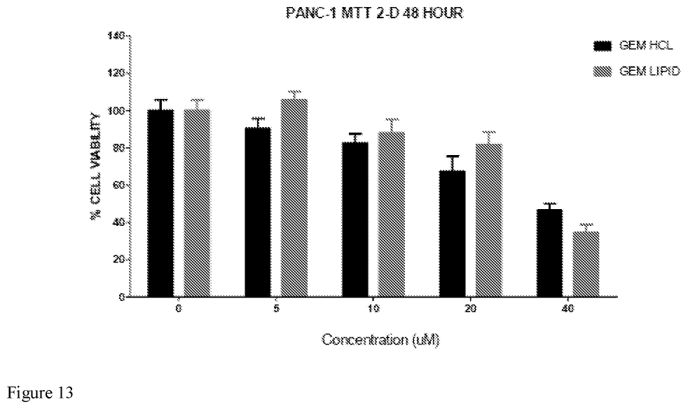

FIG. 13 is a plot of cell viability of PANC-1 cells treated with (a) gemcitabine hydrochloride (0-40 μM) and (b) DSPE-Gem (0-40 μM).

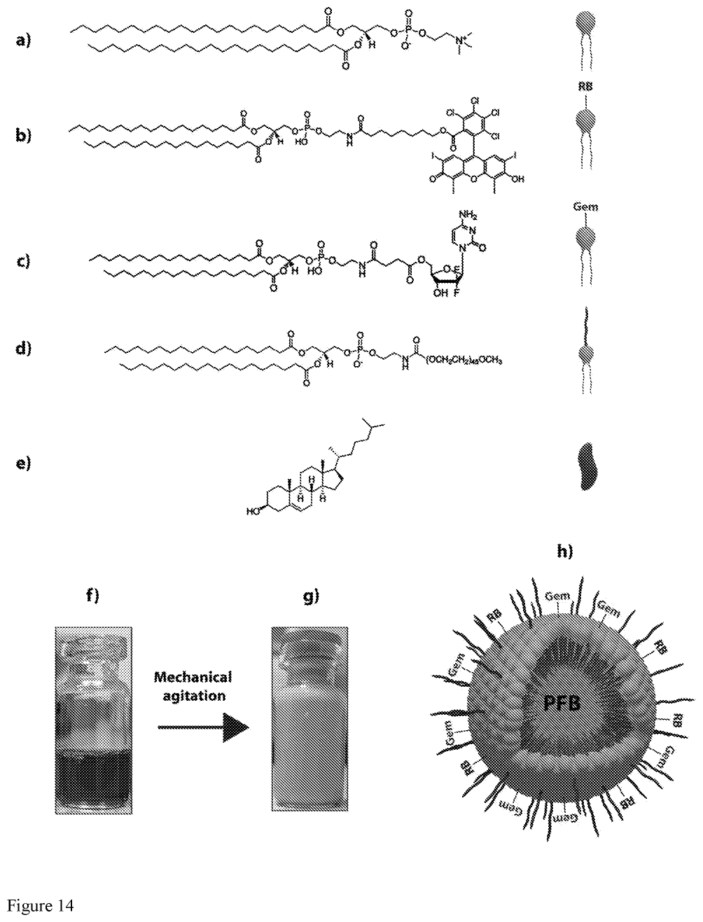

FIG. 14 shows chemical structures of (a) DSPC; (b) DSPE-RB; (c) DSPE-Gem; (d) DSPE-PEG (2000); (e) cholesterol. Images of (f) pre-microbubble lipid suspension and (g) freshly prepared microbubble suspension. 3D schematic representation of (h) Gem-RB-MB.

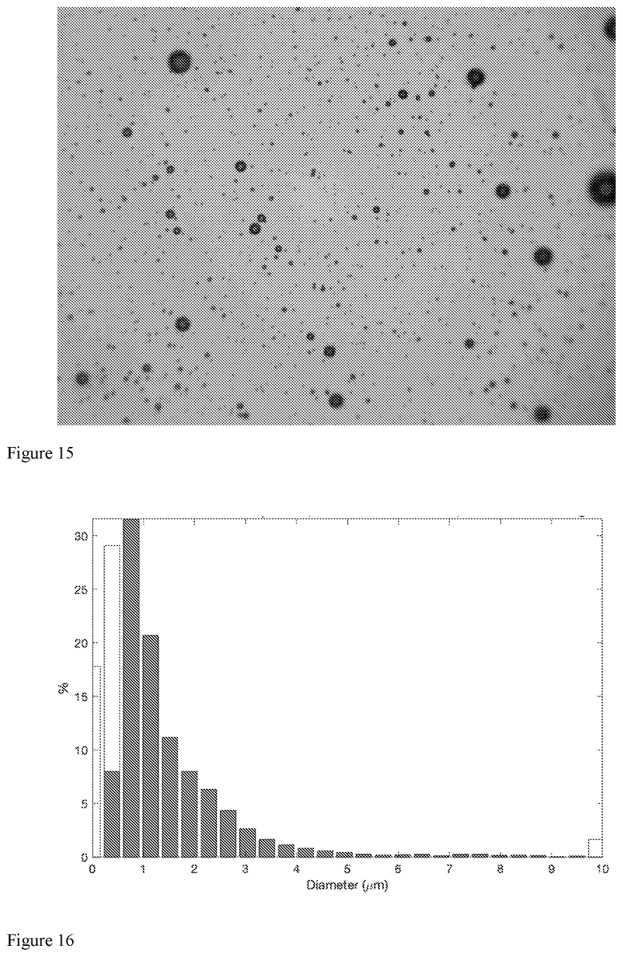

FIG. 15 is an optical microscope image of Gem-RB-MBs.

FIG. 16 is a size distribution analysis of Gem-RB-MBs.

### EXAMPLES

### Example 1—Preparation of Gemcitabine-functionalised microbubbles

1.1 Synthesis of “Lipid-Gem”

A Gemcitabine-functionalised lipid (“Lipid-Gem”) was produced by reaction of gemcitabine hydrochloride and 1,2-dibehenoyl-sn-glycero-3-phosphocholine in a biphasic emulsion of chloroform and aqueous buffer (200 mM sodium acetate, 200 mM calcium chloride). The reaction was catalysed through the addition of phospholipase D to the aqueous buffer prior to mixing.

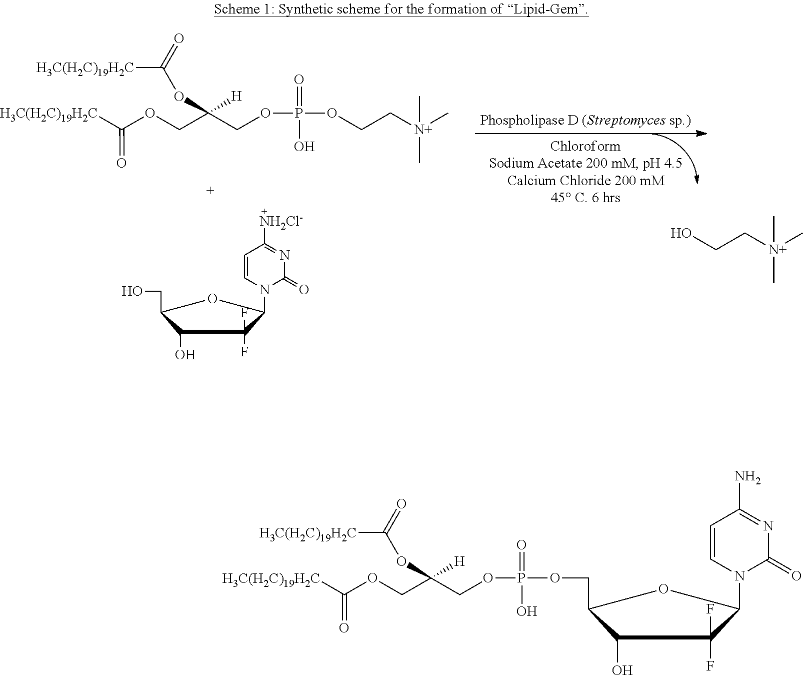

A chloroform solution (20 mL) of 1,2-dibehenoyl-sn-glycero-3-phosphocholine (500 mg, 480 μmol) was added to a stirred solution of gemcitabine hydrochloride (400 mg, 1.5 mmol) and phospholipase D from Streptomyces sp (3 mg, 900 units) in sodium acetate buffer (200 mM, pH 4.5, 5 mL) containing calcium chloride (200 mM). The mixture was stirred vigorously at 45° C. for 6 hours after which a solution containing chloroform (10 mL) and methanol (15 mL) was added. The organic layer was separated and the aqueous layer washed twice with a chloroform/methanol mixture (2:1). The organic extracts were combined, dried using anhydrous sodium sulphate, filtered and concentrated in vacuo to yield a waxy, off-white solid. The crude product was purified using preparative thin layer chromatography (chloroform:methanol:(7N) ammonium hydroxide, 65:25:4, Rf −0.41) to give “Lipid-Gem” (25% yield).

Formation of “Lipid-Gem” was confirmed by matrix-assisted laser desorption/ionization-time of flight (MALDI-TOF) spectroscopy and ¹H NMR. The MALDI-TOF spectrum of “Lipid-Gem” (m/z 500-2000) is shown in FIG. 1. The base peak at 1084.7758 corresponds to the [M+Na]⁺ ion. The peak at 1062.7800 corresponds to the [M+H]⁺ ion. The peak at 1106.7753 corresponds to the [M+2Na—H]⁺ ion. FIG. 2 shows the stacked ¹H NMR spectra of 1,2-dibehenoyl-sn-glycero-3-phosphocholine standard (top), gemcitabine standard (middle) and “Lipid-Gem” (bottom) recorded in a CDCl₃/MeOD mixed solvent system. The resonances corresponding to the choline moiety present on the phospholipid polar head group (3.2 ppm) and the neighbouring methylene protons (3.6 ppm) shown in the top spectrum are no longer present in the spectrum corresponding to “Lipid-Gem” indicating a successful cleavage of the choline group. In addition, characteristic protons from gemcitabine such as the two aromatic ring protons which appear at 5.9 and 7.8 ppm respectively and the 3′ hydroxyl group which appears at 5.2 ppm are clearly visible in the spectrum corresponding to “Lipid-Gem”.

1.2 Preparation of Perfluorobutane (PFB) Loaded “Lipid-Gem” Microbubbles

“Lipid-Gem” (5 mg, 4.71 μmol) was dissolved in a mixture of chloroform and methanol (2:1, 100 L) and then placed in a vacuum oven at 40° C. for 1 hour to allow the solvent to evaporate. The dried lipid film was rehydrated in a mixture of PBS, glycerol and propylene glycol (8:1:1, 2 mL) and stirred at 90° C. for 1 hour. The liposomal suspension was then sonicated using a probe sonicator at power setting 25% for 1 minute. The suspension was then further sonicated at power setting 90% under an atmosphere of perfluorobutane gas for 30 seconds to form a milky-white microbubble suspension. This suspension was transferred to a centrifuge tube and centrifuged (5 min, 100 rcf). The infranatant was discarded and the microbubbles were then re-suspended in a mixture of PBS, glycerol and propylene glycol (8:1:1) and used immediately.

1.3 Preparation of PFB Loaded “Lipid-Gem+Paclitaxel (PTX)” Microbubbles

“Lipid-Gem” (5 mg, 4.71 μmol), 1,2-distearoyl-sn-glycero-3-phosphoethanolamine-N-[methoxy(polyethylene glycol)-2000] (1.43 mg, 0.51 mol) and paclitaxel (2.5 mg, 2.93 μmol) were dissolved in a mixture of chloroform and methanol (2:1, 100 μL) and then placed in a vacuum oven at 40° C. for 1 hour to allow the solvent to evaporate. The dried lipid film was rehydrated in a mixture of PBS, glycerol and propylene glycol (8:1:1) and stirred at 90° C. for 1 hour. The liposomal suspension was then sonicated suing a probe sonicator at power setting 25% for 1 minute. The suspension was then further sonicated at power setting 90% under an atmosphere of perfluorobutane gas for 30 seconds to form a milky white microbubble suspension. This suspension was transferred to a centrifuge tube and centrifuged (5 min, 100 ref). The infranatant was discarded and the microbubbles were then re-suspended in a mixture of PBS, glycerol and propylene glycol (8:1:1) and used immediately.

### Example 2—Determination of Mean Diameter and Concentration of Microbubbles Produced in Example 1

A sample of freshly prepared microbubble suspension (10 μL) was diluted in PBS (990 μL) and a sample of this suspension (10 μL) was loaded into a haemocytometer chamber. Using an optical microscope (×40 objective), 20 images of the microbubbles were taken. The microbubble size distribution and concentration was then determined through image analysis using ImageJ software. The brightfield image was converted to 8-bit greyscale before an automated threshold strategy was applied. Particle diameter was then calculated relative to the scale bar present in the brightfield image.

FIG. 3 shows (a) a representative optical micrograph of the “Lipid-Gem” microbubbles (1:25 dilution, ×40 magnification); and (b) a representative size distribution analysis of images of the “Lipid-Gem” microbubbles. The mean concentration of the microbubbles was 1.8×10⁹+1.5×10⁸ microbubbles/mL. The mean diameter of the microbubbles was determined to be 2.4±1.9 μm.

FIG. 4 shows (a) a representative optical micrograph of the “Lipid-Gem+DSPE-PEG (2000)+PTX” microbubbles (1:25 dilution, ×40 magnification); and (b) representative size distribution analysis of images of “Lipid-Gem+DSPE-PEG (2000)+PTX” microbubbles. The mean concentration of the microbubbles was 1.7×10⁹+1.1×10⁸ microbubbles/mL. The mean diameter of the microbubbles was determined to be 2.3±1.7 μm.

### Example 3—In Vitro Cytotoxicity of “Lipid-Gem” Microbubbles and “Lipid-Gem+PTX” Microbubbles in Panc-1 Spheroids

The human primary pancreatic carcinoma cell line PANC-1 was maintained in Dulbecco's Modified Eagle's Medium (DMEM) containing 1 g/L glucose and supplemented with 100 U/mL penicillin, 100 mg/mL streptomycin, and 10% fetal bovine serum (FBS). Cells were incubated at 37° C. in a humidified atmosphere with 5% CO₂. Spheroids were prepared by seeding 2000 cells (200 μL) into a pre-coated 96-well plate (60 μL 1.5% agarose per well). Cells were incubated for 5 days to allow for spheroid formation. The media in each well was then replaced with either fresh media, “Lipid-Gem” microbubbles ([Lipid-Gem]=10 μM) or “Lipid-Gem+PTX” microbubbles ([Lipid-Gem]=10 μM, [PTX]=6.2 μM). Selected wells were then treated individually with ultrasound (Sonidel SP100 sonoporator, 30 s, frequency—1 MHz, ultrasound power density—3.0 W/cm², duty cycle—40%). Two days after initial treatment spheroids were washed four times with PBS. Spheroids were then placed into Eppendorf tubes and re-suspended in 90 μL of media. To this suspension was added 10 μL of MTT (5 mg/mL in PBS) and the cells incubated for 3 hours. Each tube was then centrifuged and the supernatant was discarded. The pellet was then dissolved in DMSO (100 μL) and the contents of each tube was transferred to a 96 well plate. The absorbance of the formazan dye metabolite was measured at 570 nm using a plate reader.

Results are shown in FIG. 5 by way of representative images of the Panc-1 spheroids. There was no apparent effect on spheroid morphology when the spheroids were treated with ultrasound in the absence of microbubbles. Spheroids treated with the “Lipid-Gem” microbubbles with no ultrasound stimulus also did not show any degradation in morphology. However, upon application of the ultrasound stimulus, the tightly packed spheroid structure began to show signs of degradation with small areas of cell debris becoming apparent. This effect was further amplified when PTX was added to the formulation. Significant structural damage is apparent in spheroids treated with the “Lipid-Gem+PTX” microbubbles in the absence of ultrasound stimulus. The most evidential morphological degradation of spheroids occurred upon ultrasound treatment of spheroids treated with the “Lipid-Gem+PTX” microbubbles.

FIG. 6 shows the cell viability of the Panc-1 spheroids in FIG. 5. No statistically significant cell death was observed in spheroids treated with either “Lipid-Gem” microbubbles or “Lipid-Gem+PTX” microbubbles in the absence of ultrasound. However, a statistically significant decrease in cell viability of 38% was observed for spheroids treated with “Lipid-Gem” microbubbles plus ultrasound. A further 31% decrease in cell viability was observed when spheroids were treated with “Lipid-Gem+PTX” microbubbles plus ultrasound.

### Example 4—In Vivo Cytotoxicity of “Lipid-Gem” Microbubbles and “Lipid-Gem+PTX” Microbubbles in a BxPC-3 Xenograft Model

All animals in these studies were treated humanely and in accordance with licensed protocols under the Animals (Scientific Procedures) Act 1986 (UK). BxPc-3 cells were maintained in RPMI 1640 media supplemented with 100 U/mL penicillin, 100 mg/mL streptomycin, and 10% fetal bovine serum (FBS) in a humidified 5% CO₂ atmosphere at 37° C. Cells (1×10⁶) were re-suspended in Matrigel® and implanted subcutaneously into the rear dorsum of SCID (C.B-17/IcrHan@Hsd-Prkdcscid) mice. Tumours reached treatable size within 3 weeks. Tumour measurements were taken daily using callipers. Once the tumours had reached an average volume of 150 mm³ animals were randomly assigned into treatment groups. Animals were anaesthetised using isoflurane with medical grade oxygen as the carrier gas. Animals were treated with a 100 μL I.V. injection of either “Lipid-Gem” microbubble ([Lipid-Gem]=70.1 μg/10⁸ MB) or “Lipid-Gem+PTX” microbubbles ([Lipid-Gem]=75.1 μg/10⁸ MB, [PTX]=21.3 μg/10⁸ MB). As control groups, animals were treated with a 100 μL I.P injection of either (i) gemcitabine hydrochloride (120 mg/kg) or (ii) gemcitabine hydrochloride (120 mg/kg) and PTX (15 mg/kg). Ultrasound was applied directly to the tumour immediately following injection at an ultrasound frequency of 1 MHz, an ultrasound power density of 3.5 W/cm² and a duty cycle of 30% for 3.5 min. Tumour volume was measured using callipers and calculated using the formula: V=L×W×H/2.

Results are shown in FIGS. 7 and 8. The tumour growth plot in FIG. 7 reveals that the scaled clinical dose of Gem HCl significantly controls tumour growth progression. “Lipid-Gem” microbubbles without PTX showed significant improvement in tumour growth control compared to Gem HCl. The greatly enhanced tumour inhibitory effect delivered by treatment with the “Lipid-Gem” microbubbles clearly demonstrates one of the advantages of the invention since the amount of Gem in this formulation is some 120-fold lower than that administered to the group treated with the clinical dose of Gem (i.e. Gem-HCl). There was a further small but non-significant decrease in tumour growth progression for mice treated with “Lipid-Gem+PTX” microbubbles compared to animals treated with non-PTX containing microbubbles. The plot in FIG. 8 of % change in body weight against time during treatment shows that no significant reduction in body weight was observed in any animal from any of the treatment groups. In contrast, administration of an equivalent “control” for the combination of free Gem HCl and PTX (i.e. a scaled clinical dose) was found to result in a significant drop in body weight of the animals. This confirms the microbubble delivery of Gem and PTX in accordance with the invention is improved compared to a conventional treatment and well tolerated.

### Example 5—Preparation of 5-Flourouridine-Functionalised Lipid

A 5-fluorouridine-functionalised lipid (“Lipid-5FUR”) was produced by reaction of 5-fluorouridine and 1,2-dibehenoyl-sn-glycero-3-phosphocholine in a biphasic emulsion of chloroform and aqueous buffer (200 mM sodium acetate, 200 mM calcium chloride). The reaction was catalysed through the addition of phospholipase D to the aqueous buffer prior to mixing.

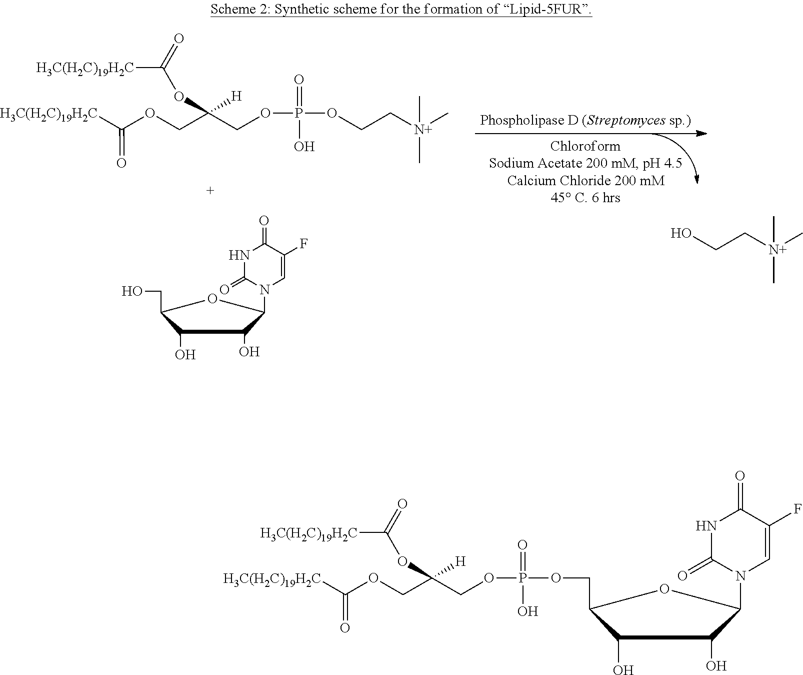

A chloroform solution (20 mL) of 1,2-dibehenoyl-sn-glycero-3-phosphocholine (500 mg, 480 μmol) was added to a stirred solution of 5-flourouridine (395 mg, 1.5 mmol) and phospholipase D from Streptomyces sp (3 mg, 900 units) in sodium acetate buffer (200 mM, pH 4.5, 5 mL) containing calcium chloride (200 mM). The mixture was stirred vigorously at 45° C. for 6 hours after which a solution containing chloroform (10 mL) and methanol (15 mL) was added. The organic layer was separated, and the aqueous layer washed twice with a chloroform/methanol mixture (2:1). The organic extracts were combined, dried using anhydrous sodium sulphate, filtered and concentrated in vacuo to yield a white solid. The crude product was purified using preparative thin layer chromatography (chloroform:methanol:(7N) ammonium hydroxide, 65:25:4) to give “Lipid-5FUR”.

FIG. 9 shows the stacked ¹H NMR spectra of 1,2-dibehenoyl-sn-glycero-3-phosphocholine standard (top), 5-flourouridine standard (middle) and Lipid-5FUR (bottom). The resonances corresponding to the choline moiety present on the phospholipid polar head group (3.2 ppm) and the neighbouring methylene protons (3.6 ppm) shown in the top spectra are no longer present in the spectra corresponding to “Lipid-5FUR” indicating a successful cleavage of the choline group. In addition, characteristic protons from the uracil moiety of 5-flourouridine such as the imide proton which appears at 8.0 ppm and the aromatic ring proton which appears at 5.9 ppm are clearly visible in the spectra corresponding to “Lipid-5FUR”. FIG. 10 is a matrix-assisted laser desorption/ionization-time of flight (MALDI-TOF) spectra of “Lipid-5FUR” (m/z 199-2000) in negative mode. The base peak at 1069.48 corresponds to the [M−H]⁻ ion. The peak at 603.30 peak is from the matrix.

### Example 6—Preparation of Gemcitabine-Functionalised Lipid

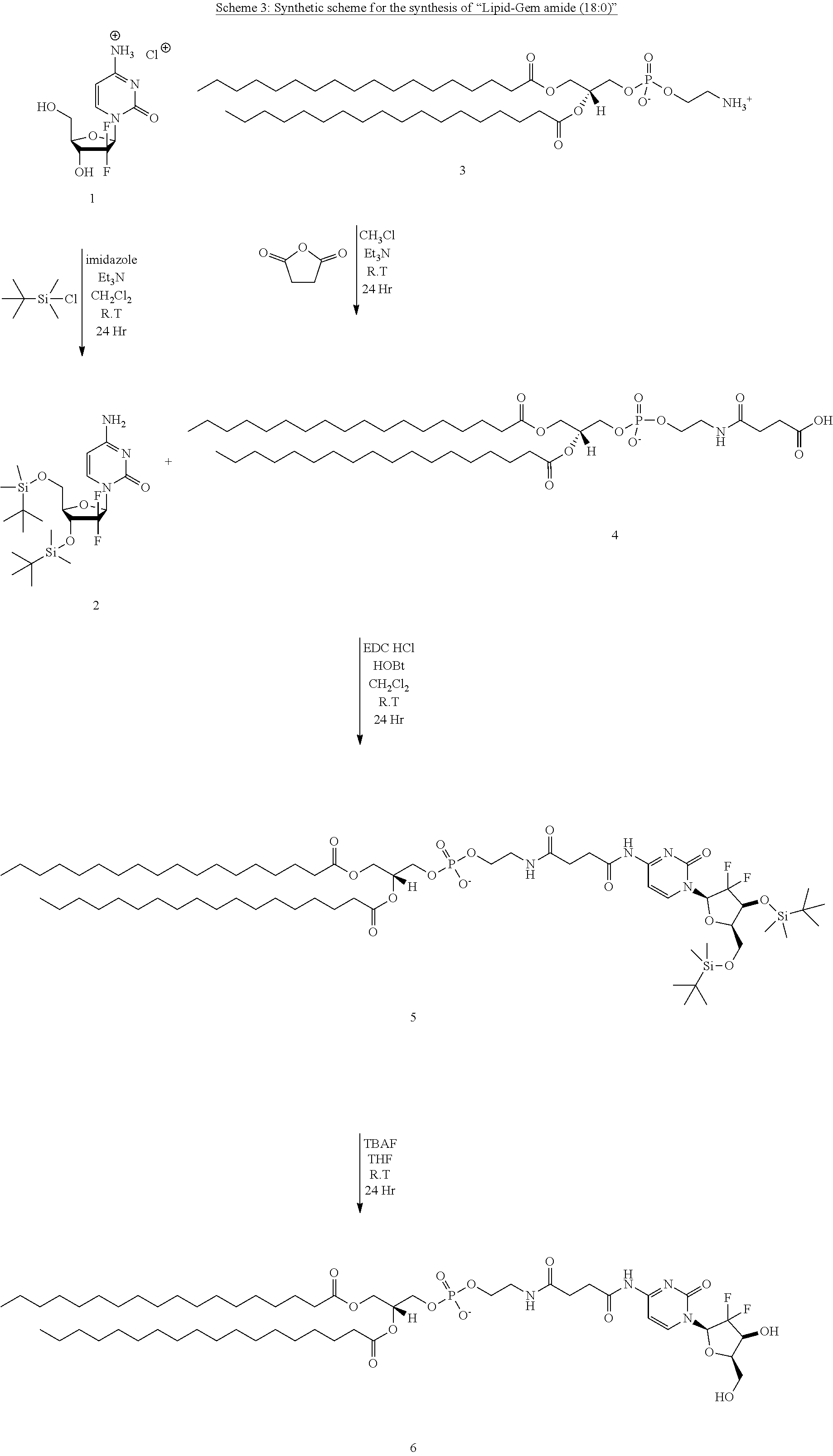

1,2-distearoyl-sn-glycero-3-phosphoethanolamine (3) was used as the commercially available starting material. The primary amine of 3 was reacted with succinic anhydride in basic conditions to form an amide bond and terminal carboxylic acid group. In parallel, the 3- and 5-hydroxyl functionalities of 1 were protected with tert-butyl dimethyl silyl ethers by reacting 1 with TBDSM-Cl and imidazole in basic conditions. This was followed by an amidation reaction between the primary amine group of 2 and the pendant carboxylic acid of 4 to form 5. The tert-butyl dimethyl silyl ethers of the gemcitabine moiety of 5 were then deprotected using TBAF to yield “Lipid-Gem amide (18:0)”.

6.1 Synthesis of TBDMS-Gem (2)

To a stirred solution of gemcitabine hydrochloride (1, 1 g, 3.4 mmol), imidazole (2.32 g, 34 mmol) and TBDMS-Cl (5.3 g, 20 mmol) in anhydrous dimethylformamide (50 mL) was added dropwise triethylamine (1.12 mL, 6.8 mmol). The solution was stirred at room temperature under a nitrogen atmosphere for 24 hours before which the solvent was removed in vacuo. The residue was taken up in aqueous sodium hydrogen carbonate (10% w/v) solution and extracted with ethyl acetate (5×100 mL). The organic extracts were combined and washed with aqueous sodium hydrogen carbonate (10% w/v) solution (1×50 mL) and brine (2×50 mL) before the solvent was removed in vacuo. The crude solid was purified using column chromatography (dichloromethane:methanol 9:1) to give 2 as a white crystalline solid (1.5 g, 91%).

6.2 Synthesis of N-succinyl DSPE (4)

To a stirred solution of DSPE (3, 0.9 g, 1.2 mmol) and triethylamine (669 μL, 4.8 mmol) in chloroform (40 mL) was added succinic anhydride (0.15 g, 1.4 mmol). The solution was stirred under a nitrogen atmosphere for 24 hours after which the solvent was removed in vacuo. The crude residue was precipitated in ice cold acetone, filtered and washed thoroughly with ice cold acetone to give 4 (0.8 g 78%).

6.3 Synthesis of TBDMS-Gem-N-DSPE (5)

To a stirred solution of 4 (0.7 g, 0.8 mmol) in anhydrous dichloromethane (40 mL) was added EDC HCl (0.29 g, 1.5 mmol) and HOBt (0.2 g, 1.5 mmol). This solution was stirred at room temperature under a nitrogen atmosphere for 30 minutes after which time 2 (0.43 g, 0.88 mmol) was added. The solution was stirred for a further 24 hours after which time the solvent was removed in vacuo. The residue was precipitated in ice cold acetone, filtered and washed thoroughly with ice cold acetone to yield 5 (0.6 g). This was used in the next step without further purification.

6.4 Synthesis of DSPE-Gem (6)

To a stirred suspension of 5 (0.6 g, 0.45 mmol) in anhydrous tetrahydrofuran (20 mL) in an ice bath was added dropwise TBAF (1M in THF, 1.13 mL, 1.13 mmol). The suspension slowly dissolved over the course of 15 minutes and the ice bath was removed. The solution was stirred for a further 24 hours before the solvent was removed in vacuo. The residue was precipitated in ice cold acetone, filtered and washed thoroughly in ice cold acetone. The crude product was purified using preparative thin layer chromatography (chloroform:methanol:ammonium hydroxide (7N) 65:25:4) to give 6 (60% yield for last two steps).

FIG. 11 is a matrix-assisted laser desorption/ionization-time of flight (MALDI-TOF) spectra of “Lipid-Gem amide (18:0)” (m/z 199-2000) in negative mode. The base peak at 1090.33 corresponds to the [M−H]⁻ ion. FIG. 12 shows the ¹H NMR spectra of “Lipid-Gem amide (18:0)”. The characteristic aromatic protons from the cytosine moiety of gemcitabine appear as two doublets at 6.10 and 8.15 ppm. The two sets of protons from the ethyl linker appear at 2.4 and 2.65 ppm.

### Example 7—Preparation of Gemcitabine-Functionalised Lipid

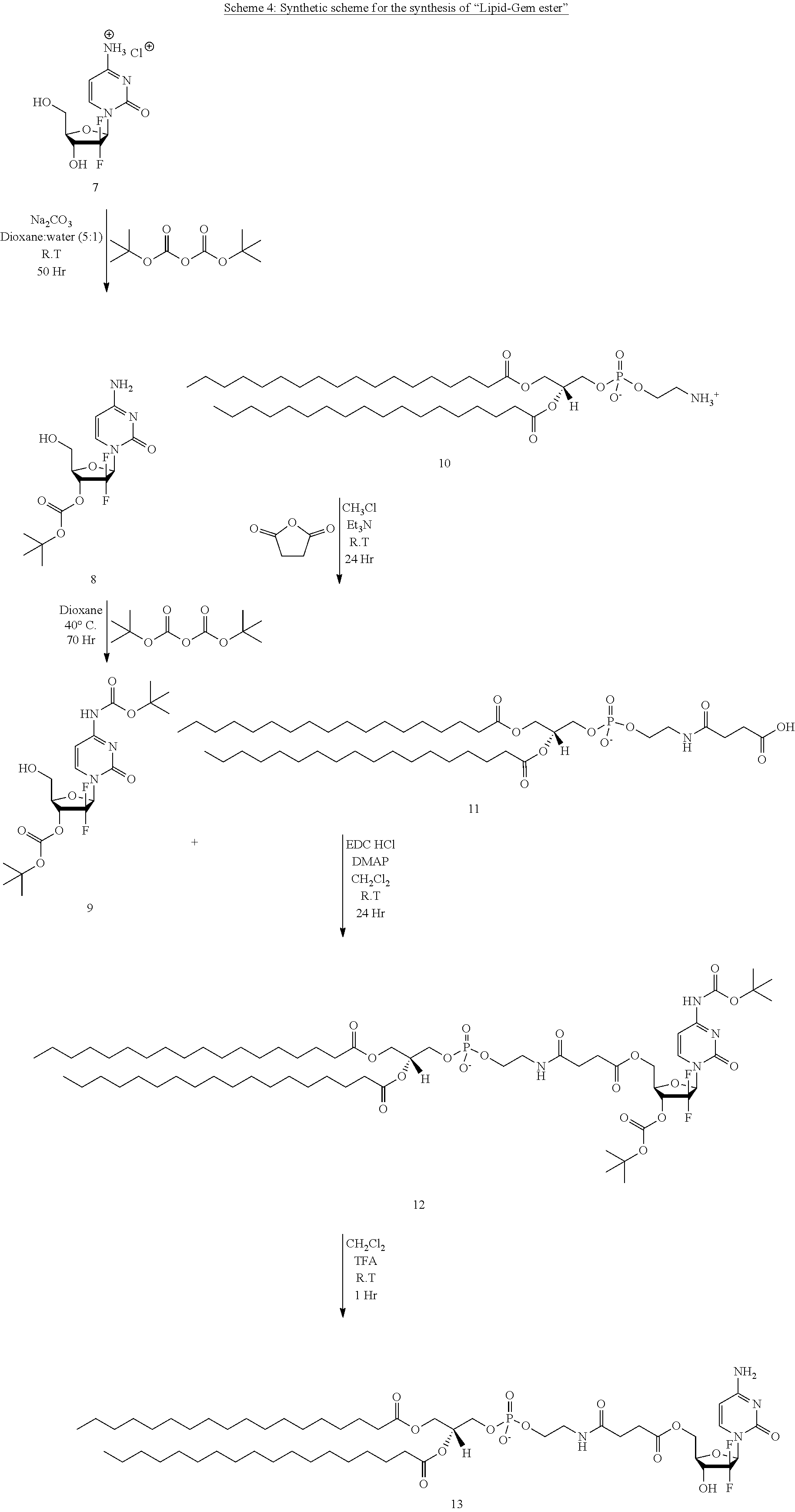

An alternative gemcitabine-functionalised lipid may be prepared according to Scheme 4 above. This scheme utilises 1,2-distearoyl-sn-glycero-3-phosphoethanolamine (10) as the commercially available starting material. The primary amine of 10 is reacted with succinic anhydride in basic conditions to form an amide bond and terminal carboxylic acid group. In parallel, the 3-hydroxyl and primary amine functionalities of 7 are protected with tert-butyl carbonate esters by reacting 7 successively with DBDC. This is followed by an esterification reaction between the 5-hydroxyl group of 9 and the pendant carboxylic acid of 11 to form 12. The tert-butyl carbonate esters of the gemcitabine moiety of 12 are then deprotected using TFA to yield “Lipid-Gem ester (18:0)” (13).

### Example 8—Preparation of Gemcitabine-Functionalised Lipid

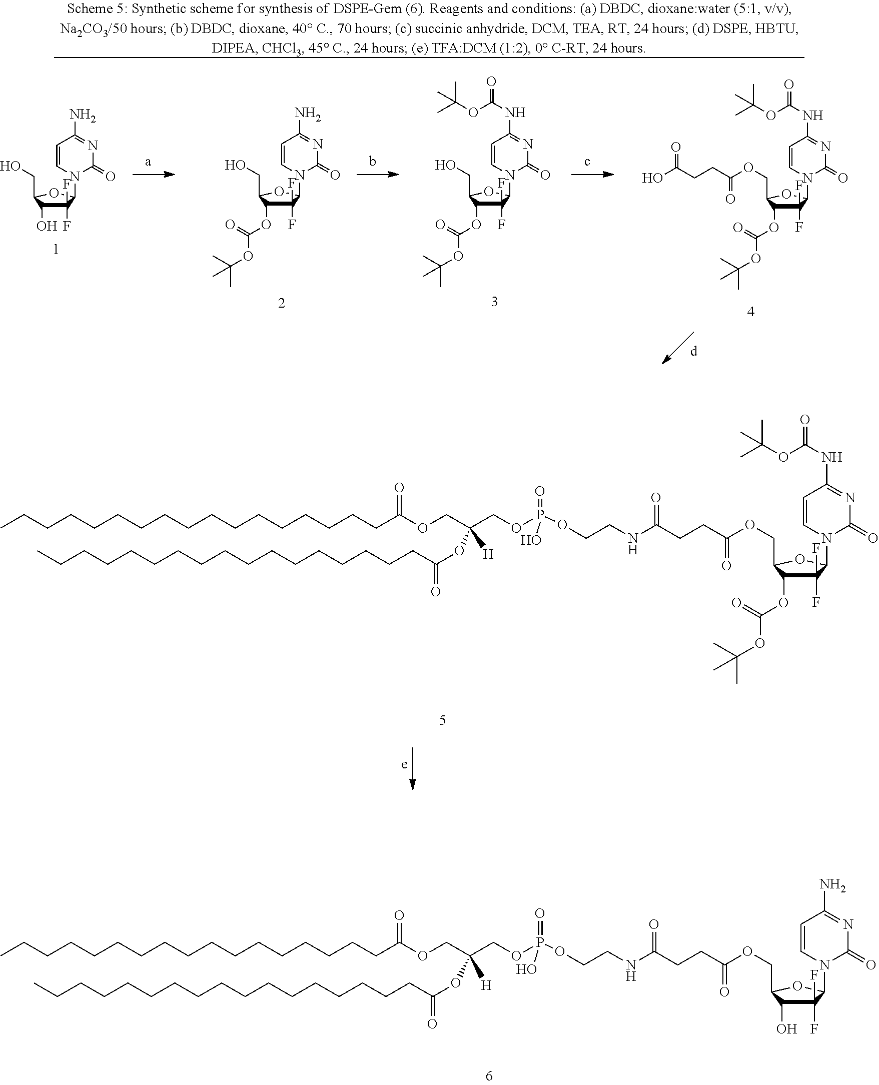

### 8.1 Synthesis of (2R,3R,5R)-5-(4-amino-2-oxopyrimidin-1(2H)-yl)-4,4-difluoro-2-(hydroxymethyl)tetrahydrofuran-3-yl tert-butyl carbonate (2)

Di-tert-butyl dicarbonate (DBDC) (0.36 g, 1.65 mmol) was added to the solution of gemcitabine hydrochloride (1) (0.50 g, 1.67 mmol) and Na₂CO₃ (2.40 g, 8.49 mmol) in a mixture of dioxane and water (5:1, v/v, 10 ml) and the reaction was stirred at room temperature for 50 hours. Subsequent TLC analysis (DCM:acetone:ethanol, 5:4:1, v/v) showed almost complete consumption of gemcitabine. The reaction mixture was then diluted with water (100 mL) and extracted with ethyl acetate (3×30 mL). The combined organic extracts were washed with brine (20 mL), dried over sodium sulfate, filtered and concentrated to dryness under reduced pressure. The residue was washed with diethyl ether to afford compound 2 as a white solid (0.57 g, 95%). ¹H NMR (DMSO-d₆): 7.65 (d, J=7.5 Hz, 1H, —CH), 7.39 (brs, 2H, —NH₂), 6.15 (s, 1H, —CH), 5.91 (d, J=7. Hz, 1H, —CH), 5.19 (brs, 1H, —CH), 5.09 (brs, 1H, —OH), 4.09 (d, J=5.5 Hz, 1H, —CH), 3.89-3.79 (m, 2H, —CH₂), 1.42 (s, 9H, —CH₃×3). ESI-MS: calculated for C₁₄H₁₉F₂N₃O₆=363.3; found=386.1 [M+Na]⁺.

### 8.2 Synthesis of tert-butyl(1-((2R,4R,5R)-4-((tert-butoxycarbonyl)oxy)-3,3-difluoro-5-(hydroxymethyl) tetrahydrofuran-2-yl)-2-oxo-1,2-dihydropyrimidin-4-yl)carbamate (3)

DBDC (3.00 g, 13.74 mmol) was added to a stirred solution of 2 (0.50 g, 1.38 mmol) in dioxane (25 mL) and the resulting mixture was stirred at 40° C. for 70 hours. Subsequent TLC analysis (DCM:Acetone:EtOH, 5:4:1, v/v) showed complete consumption of compound 2. The solvent was removed under reduced pressure and the residue was suspended in water (10 mL) and extracted with DCM (4×20 mL). The combined organic extracts were washed with brine (10 mL), dried over sodium sulfate, filtered and concentrated to dryness under reduced pressure. The crude compound was purified by column chromatography (DCM:Acetone, 5%-20%, v/v) to afford compound 3 as a white powder (0.41 g, 65%). ¹H NMR (CDCl₃:MeOH, 2:1 v/v) δ: 8.12 (d, J=7.5 Hz, 1H, —CH), 7.35 (d, J=7.5 Hz, 1H, —CH), 6.39-6.29 (m, 1H, —CH), 5.19-5.11 (m, 1H, —CH), 4.18 (d, J=5.5 Hz, 1H, —CH), 3.99-3.79 (m, 2H, —CH₂), 1.45 (s, 18H, CH₃×6). ¹³C NMR (CDCl₃:MeOH, 2:1 v/v): 164.0 (C), 154.3 (CO), 152.3 (CO), 151.6 (CO), 145.1 (CH), 121.8 (C), 95.5 (CH), 84.2 (C), 81.7 (C), 79.3 (CH), 59.5 (CH₂), 28.1 (CH₃), 27.5 (CH₃). ESI-MS: Calculated for C₁₉H₂₇F₂N₃O₈=463.4; found=486.3 [M+Na]⁺.

### 8.3 Synthesis of 4-(((2R,3R,5R)-5-(4-((tert-butoxycarbonyl)amino)-2-oxopyrimidin-1(2H)-yl)-3-((tert-butoxycarbonyl)oxy)-4,4-difluorotetrahydrofuran-2-yl)methoxy)-4-oxobutanoic acid (4)

To a stirred solution of 3 (1.63 g, 3.5 mmol) in anhydrous DCM (15 mL) was added TEA (1.42 g, 14.0 mmol) and this solution was stirred at room temperature for 10 minutes followed by the addition of succinic anhydride (0.70 g, 7.00 mmol). This solution was stirred at room temperature for 24 hours or until no starting material was visible by TLC (DCM:acetone:acetic acid, 6:2:0.05 v/v). Following completion of the reaction the solution was washed with distilled water (3×20 mL) and brine (3×20 mL) and dried with anhydrous sodium sulphate before removal of the solvent under reduced pressure. The resultant crude product was purified using column chromatography (DCM:acetone:acetic Acid, 6:2:0.05 v/v) to afford compound 4 as an off-white crystalline solid (1.65 g, 82%). ¹H NMR (DMSO-d6) δ: 12.25 (brs, 1H, —COOH), 10.51 (brs, 1H, —NH), 8.01 (d, J=7.5 Hz, 1H, —CH), 7.05 (d, J=7.5 Hz, 1H, —CH), 6.13 (m, 1H, —CH), 5.13 (brs, 1H, —CH), 4.55-4.15 (m, 3H, —CH, —CH₂), 2.61-2.33 (m, 4H, —CH₂×2), 1.55 (s, 18H, —CH₃×6). ¹³C NMR (CDCl₃:MeOH, 2:1 v/v): 164.0 (CO), 154.3 (CO), 152.3 (C), 151.6 (CO), 145.1 (CH), 121.8 (C), 95.5 (CH), 84.2 (C), 81.7 (CH), 79.3 (CH), 59.5 (CH₂), 39.6 (CH₂), 28.1 (CH₃), 27.5 (CH₃). ESI-MS: Calculated for C₂₃H₃₁F₂N₃O₁₁=563.5; found=562.0 [M−H]⁻.

### 8.4 Synthesis of 3-(((2-(4-(((2R,3R,5R)-5-(4-amino-2-oxopyrimidin-1(2H)-yl)-4,4-difluoro-3-hydroxytetrahydrofuran-2-yl)methoxy)-4-oxobutanamido)ethoxy)(hydroxy)phosphoryl)oxy) propane-1,2-diyl distearate (6)

To a stirred solution of DSPE (0.10 g, 0.13 mmol), 4 (0.09 g, 0.15 mmol) and HBTU (0.06 g, 0.15 mmol), in chloroform (15 mL), was added DIPEA (0.02 g, 0.15 mmol) and the solution was stirred under reflux at 45° C. for 24 hours until no starting material was visible by TLC (CHCl₃:CH₃OH, 8:2 v/v). After completion of the reaction the solvent was removed under reduced pressure to yield the resultant crude compound 5 (0.175 g, 0.13 mmol). To a solution of 5 in anhydrous DCM (10 mL) was added TFA (5 mL) and the resulting solution was stirred at 0° C. for 1 hour followed by further stirring at room temperature for 24 hours until no starting material was visible by TLC (CHCl₃:CH₃OH, 8:2 v/v). Following completion of the reaction, the solvent was removed under reduced pressure and the residue was dissolved in CHCl₃:CH₃OH (2:1 v/v) and washed with an aqueous sodium bicarbonate solution (10% w/v). The organic layer was separated and concentrated under reduced pressure. The resultant crude product was purified using column chromatography (DCM:MeOH 2:1 v/v) to afford compound 6 as a white powder (0.09 g, 61%). ¹H NMR (CDCl₃:MeOH, 2:1) 6:7.53 (d, J=7.5 Hz, 1H, —CH), 6.28-6.25 (m, 1H, —CH), 6.01 (d, J=7.5 Hz, 1H, —CH), 5.24-5.22 (m, 1H, —CH), 4.45-4.39 (m, 4H, —CH×2, —CH₂), 4.19-3.89 (m, 6H, —CH₂×3), 3.40 (t, 2H, —CH₂), 2.73-2.67 (m, 2H, —CH₂), 2.59-2.56 (m, 2H, —CH₂), 2.33-2.29 (m, 4H, —CH₂×2), 1.60-1.59 (m, 4H, —CH₂×2), 1.28-1.26 (m, 56H, —CH₂×28), 0.87 (t, 6H, —CH₃×2). ¹³C NMR (CDCl₃:MeOH, 2:1v/v): 173.9 (CO), 173.5 (CO), 173.4 (CO), 172.2 (CO), 165.9 (C), 156.3 (CO), 140.7 (CH), 124.0 (C), 121.8 (CH), 95.7 (CH), 84.0 (CH), 78.3 (CH), 70.3 (CH), 69.9 (CH₂), 70.3 (CH₂), 63.8 (CH₂), 63.3 (CH₂), 62.5 (CH₂), 62.4 (CH₂), 61.7 (CH₂), 40.3 (CH₂), 34.1 (CH₂), 33.8 (CH₂), 31.8 (CH₂), 31.6 (CH₂), 30.2 (CH₂), 29.5 (CH₂), 29.4 (CH₂), 29.2 (CH₂), 29.0 (CH₂), 28.8 (CH₂), 24.7 (CH₂), 24.6 (CH₂), 22.5 (CH₂), 22.3 (CH₂), 13.7 (CH₃), 13.6 (CH₃). MALDI-TOF-MS: Calculated for C₅₄H₉₅F₂N₄O₁₄P=1093.34; found=1091.45 [M−H]⁻.

### Example 9—Preparation of Rose Bengal-Functionalised Lipid

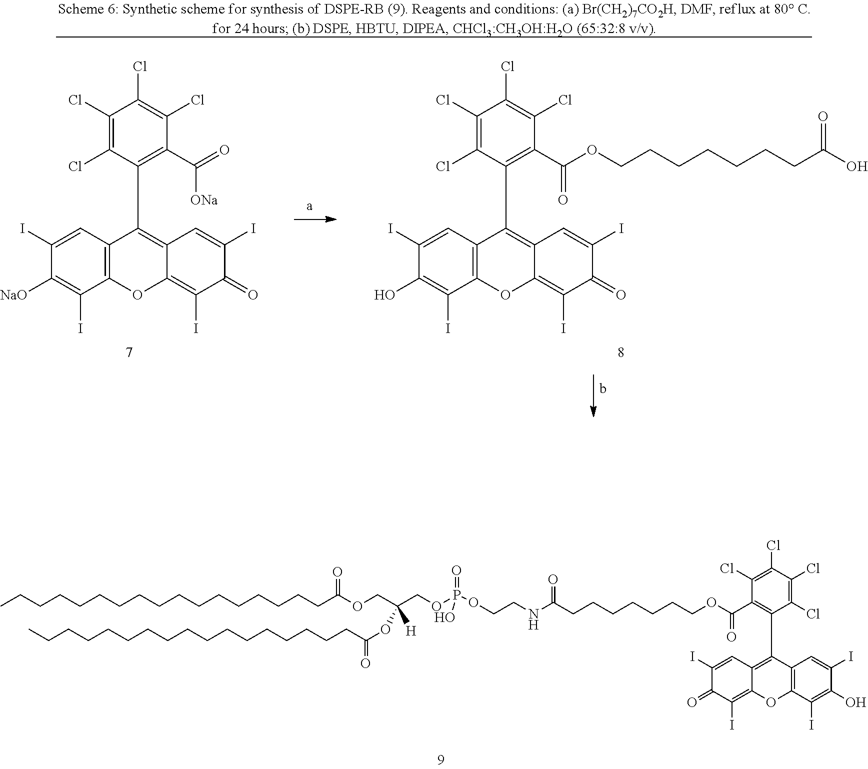

### 9.1 Synthesis of 8-((2,3,4,5-tetrachloro-6-(6-hydroxy-2,4,5,7-tetraiodo-3-oxo-3H-xanthen-9-yl)benzoyl)oxy)octanoic acid (7)

To a stirred solution of Rose Bengal disodium salt (10.00 g, 9.80 mmol) in anhydrous DMF (100 mL) was added 8-bromooctanoic acid (2.20 g, 9.80 mmol) and the solution was stirred at 80° C. for 8 hours or until no starting material was visible by TLC (CHCl₃:CH₃OH 8:2 v/v). Following completion of the reaction, the solvent was removed under reduced pressure and the residue was stirred in diethyl ether (200 mL) for 24 hours after which time the suspension was filtered and the solids were stirred in distilled water (200 mL) for 24 hours. This suspension was then filtered and dried to afford the carboxylic acid derivative 8 as a deep purple powder (8.70 g, 80%). ¹H NMR (DMSO-d₆) δ: 7.49 (s, 2H, aromatic-CH×2), 3.89 (brs, 2H, O—CH₂—), 2.09 (brs, 2H, —CH₂—COOH), 1.39 (brs, 2H, —CH₂—), 1.21-1.12 (m, 6H, —CH₂—×3), 0.89 (brs, 2H, —CH₂—). ESI-MS: Calculated for C₂₈H₁₈Cl₄I₄O₇=1115.86; found=1114.47 [M−H]⁻.

### 9.2 Synthesis of 3-((hydroxy(2-(8-((2,3,4,5-tetrachloro-6-(6-hydroxy-2,4,5,7-tetraiodo-3-oxo-3H-xanthen-9-yl)benzoyl)oxy)octanamido)ethoxy) phosphoryl)oxy)propane-1,2-diyl distearate (8)

To a stirred solution of DSPE (0.50 g, 0.60 mmol), 8 (0.90 g, 0.80 mmol) and HBTU (0.30 g, 0.8 mmol) in CHCl₃:CH₃OH:H₂O (65:35:8 v/v) was added DIPEA (0.43 g, 3.3 mmol) and the solution was stirred at room temperature for 24 hours or until no starting material was visible by TLC (CHCl₃:CH₃OH 8:2 v/v). After completion of the reaction the solvent was removed under reduced pressure and the resultant crude product was purified using column chromatography (CHCl₃:CH₃OH 8:2 v/v) to afford 9 as a deep purple powder (0.63 g, 43%). ¹H NMR (CDCl₃:CD₃OD, 2:1 v/v) δ: 7.74 (s, 2H, aromatic-CH×2), 5.25-5.23 (m, 1H, —CH), 4.19-4.15 (m, 2H, —OCH₂—), 4.01-3.95 (m, 6H, —CH₂×3), 3.53-3.51 (m, 2H, CH₂), 3.36-3.35 (m, 2H, —CH₂), 2.35-2.23 (m, 6H, —CH₂×3), 1.61-1.50 (m, 6H, —CH₂×3), 1.36-1.08 (m, 62H, —CH₂×31), 0.90-0.87 (m, 6H, —CH₃×2). ¹³C NMR (CDCl₃:CD₃OD, 2:1): 175.8 (CO), 173.9 (CO), 173.5 (CO), 173.2 (C), 163.3 (CO), 157.8 (CH), 150.8 (C), 141.5 (C), 136.8 (C), 135.6 (C), 132.1 (C), 130.3 (C), 129.5 (C), 112.8 (C), 101.6 (C), 96.2 (C), 80.1 (C), 79.3 (C), 77.0 (CH₂), 75.9 (CH₂), 72.0 (CH₂), 49.4 (CH₂), 49.0 (CH₂), 40.0 (CH₂), 36.0 (CH₂), 34.1 (CH₂), 33.9 (CH₂), 31.7 (CH₂), 31.3 (CH₂), 29.5 (CH₂), 29.2 (CH₂), 29.0 (CH₂), 28.6 (CH₂), 28.0 (CH₂), 25.0 (CH₂), 24.7 (CH₂), 22.5 (CH₂), 13.7 (CH₃). MALDI-TOF-MS: Calculated for C₆₉H₉₈Cl₄I₄NO₁₄P=1845.93; found=1844.31 [M−H]⁻.

### Example 10—Determination of the Efficacy of DSPE-Gem in PANC-1 Cells

PANC-1 cells were maintained in high glucose Dulbecco's Modified Eagle's Medium (DMEM) supplemented with 10% fetal bovine serum and incubated at 5% CO2 at 37° C. Cells were seeded (3×10³) in a 96 well plate, the following day cells were treated with several concentrations of gemcitabine hydrochloride or DSPE-Gem prepared according to Example 8 (0, 5, 10, 20 and 40 μM). Cell viability was determined 48 hours later using an MTT assay. Results are shown in FIG. 13.

### Example 11—Preparation and Characterisation of Rose Bengal and Gemcitabine Loaded Microbubbles (“RB-Gem-MB”)

Preparation of RB-Gem-MB:

To a 5 mL round bottom flask was added DBPC (1.02 mg, 1.13 μmol), DSPE-Gem prepared according to Example 8 (1.03 mg, 0.94 μmol), DSPE-RB prepared according to Example 9 (1.74 mg, 0.94 μmol), DSPE-PEG-2000 (1.06 mg, 0.38 μmol) and cholesterol (0.15 mg, 0.38 μmol) dissolved in a mixture of chloroform and methanol (2:1 v/v) to achieve a total lipid concentration of 5 mg/mL and a molar ratio of 3:2.5:2.5:10:10. The solvent was then removed using a rotary evaporator (45° C., speed setting 10) to yield a thin lipid film. Further drying of the film was carried out by placing the flask in a vacuum oven (20° C.) for 2 hours. The dried film was then hydrated by adding 1 mL of a mixture of PBS, glycerol and propylene glycol (8:1:1 v/v) (PGP) and agitating the suspension on a rotary evaporator (85° C., 45 min) in the dark. The resultant turbid lipid suspension was then sonicated using a probe sonicator (amplitude 25%, 30 seconds) to yield a transparent suspension of lipid vesicles. Once cooled to room temperature the suspension (1 mL) was added to a 3 mL crimp vial and the headspace of the vial was replaced with PFB gas and sealed. The vials were then mechanically agitated using a Vialmix device (45 seconds) to produce a purple suspension of RB-Gem-MBs. A 3D representation of the resulting “RB-Gem-MB” is shown in FIG. 14.

Characterisation of RB-Gem-MB:

An appropriate volume of freshly prepared MB-RB-Gem was diluted in cold PGP. A 10 μL sample was then loaded into the viewing chamber of a haemocytometer and viewed under a microscope (×40 objective). Microscope images (n=20) were collected and saved as high resolution TIFF files. These images were then analysed using a bespoke MATLAB algorithm to give the distribution of MB diameters and mean MB concentration. Results are shown in FIGS. 15 and 16.

1. A method of preparing a microbubble covalently attached to at least one therapeutic agent, said method comprising the following steps:
providing a lipid capable of forming a microbubble;
covalently linking at least one therapeutic agent to said lipid whereby to produce a functionalised lipid; and
preparing a microbubble from said functionalised lipid.

2. A method as claimed in claim 1, wherein said lipid is selected from the group consisting of phospholipids, fatty acids, triglycerides, diglycerides, monoglycerides, sterols and sterol derivatives, sphingolipids, and combinations thereof.

3. A method as claimed in claim 1, wherein said lipid is a phospholipid, preferably a phospholipid selected from any of the following:
phosphatidylcholines, e.g. dimyristoylphosphatidylcholine, dipalmitoylphosphatidylcholine, distearoylphosphatidylcholine, dioleoylphosphatidylcholine, dipentadecanoylphosphatidylcholine, dilauroylphosphatidylcholine, and 1,2-dibehenoyl-sn-glycero-3-phosphocholine (DBPC);
phosphatidic acids;
phosphatidylethanolamines, e.g. dioleoylphosphatidylethanolamine, and 1,2-distearoyl-sn-glycero-3-phosphoethanolamine (DSPE);
phosphatidylserines;
phosphatidylglycerols, e.g. diphosphatidylglycerols such as cardiolipin; and
phosphatidylinositols.

4. A method as claimed in claim 3, wherein said phospholipid is a compound of formula (I), or a pharmaceutically acceptable salt thereof: 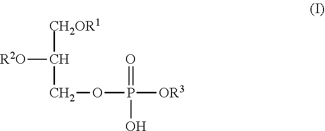
wherein:
R¹ and R², which may be the same or different, are either saturated, or mono- or polyunsaturated, C₁₀₋₃₀ acyl groups, for example —CO—C₁₀₋₂₅ alkyl or —CO—C₁₀₋₂₅ alkenyl groups; and
R³ is

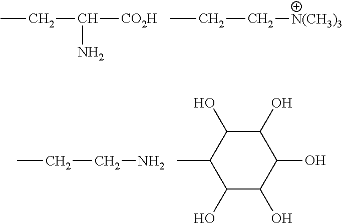

5. A method as claimed in claim 3, wherein said phospholipid is selected from 1,2-dibehenoyl-sn-glycero-3-phosphocholine (DBPC), 1,2-distearoyl-sn-glycero-3-phosphoethanolamine (DSPE), and combinations thereof.

6. A method as claimed in any one of the preceding claims, wherein the microbubble is prepared from a mixture of said functionalised lipid and one or more other lipids which are linked to one or more biocompatible polymers or polysaccharides, e.g. polyethylene glycol (PEG).

7. A method as claimed in any one of the preceding claims, wherein said functionalised lipid is produced by enzyme-catalysed phosphatidylation of a hydroxyl-containing therapeutic agent.

8. A method as claimed in claim 7, wherein said functionalised lipid is a compound of formula (II), or a pharmaceutically acceptable salt thereof: 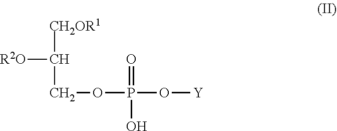
wherein
R¹ and R², which may be the same or different, are as defined in claim 4; and
Y is the residue of a therapeutic agent.

9. A method as claimed in any one of claims 1 to 6, wherein the step of covalently linking at least one therapeutic agent to said lipid whereby to produce said functionalised lipid comprises the following steps: chemical modification of said lipid to form a modified lipid; and subsequent covalent linkage of said modified lipid to said therapeutic agent.

10. A method as claimed in claim 9, wherein said lipid is a phospholipid and the step of chemical modification of said lipid comprises reacting the lipid with succinic anhydride, or a derivative thereof.

11. A method as claimed in claim 10, wherein said functionalised lipid is a compound of formula (III), or a pharmaceutically acceptable salt thereof: 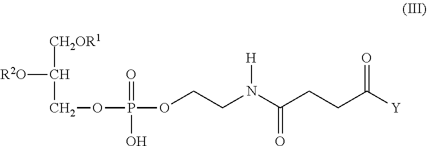
wherein
R¹ and R², which may be the same or different, are as defined in claim 4; and
Y is the residue of a therapeutic agent.

12. A method as claimed in any one of the preceding claims, wherein the step of preparing a microbubble from said functionalised lipid comprises high speed mixing or sonication of an aqueous solution comprising said functionalised lipid and one or more stabilising agents.

13. A method as claimed in any one of the preceding claims, wherein said therapeutic agent is selected from a chemotherapeutic agent, a sonosensitising agent, and a combination of such agents.

14. A method as claimed in claim 13, wherein said chemotherapeutic agent is selected from the following: antifolates (e.g. methotrexate); 5-fluoropyrimidines (e.g. 5-fluorouracil or 5-fluorouridine); cytidine analogues (e.g. gemcitabine); purine antimetabolites (e.g. mercaptopurine); alkylating agents (e.g. cyclophosphamide); non-classical alkylating agents (e.g. dacarbazine); platinum analogues (e.g. cisplatin); antitumour antibiotics (e.g. actinomycin D, bleomycin, mitomycin C); bioreductive drugs (e.g. mitomycin C, Banoxantrone (AQ4N)); anthracyclines (e.g. doxorubicin, mitoxantrone); topoisomerase I inhibitors (e.g. irinotecan); topoisomease II inhibitors (e.g. etoposide); antimicrotubule agents such as vinca alkaloids (e.g. vincristine), taxols (e.g. paclitaxel), and epothilones (e.g. ixabepiline); antioestrogens (e.g. tamoxifen); antiandrogens (e.g. biclutamide, cyproterone acetate); aromatase inhibitors (e.g. anastrazole, formestan); antiangiogenic or hypoxia targeting drugs (either naturally occuring, e.g. endostatin, or synthetic, e.g. gefitinib, lenalidomide); antivascular agents (e.g. cambretastatin); tyrosine kinase inhibitors (e.g. gefitinib, erlotinib, vandetanim, sunitinib); oncogene or signalling pathway targeting agents (e.g. tipfarnib, lonafarnib, naltrindole, rampamycin); agents targeting stress proteins (e.g. geldanamycin and analogues thereof); autophagy targeting agents (e.g. chloroquine); proteasome targeting agents (e.g. bortezomib); telomerase inhibitors (targeted oligonucleotides or nucleotides); histone deacetylase inhibitors (e.g. trichostatin A, valproic acid); DNA methyl transferase inhibitors (e.g. decitabine); alkyl sulfonates (e.g. busulfan, improsulfan and piposulfan); aziridines (e.g. benzodopa, carboquone, meturedopa, and uredopa); ethylenimines and methylamelamines (e.g. altretamine, triethylenemelamine, trietylenephosphoramide, triethylenethiophosphaoramide and trimethylolomelamine); nitrogen mustards (e.g. chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard); nitrosureas (e.g. carmustine, chlorozotocin, fotemustine, lomustine, nimustine, ranimustine); purine analogues (e.g. fludarabine, 6-mercaptopurine, thiamiprine, thioguanine); pyrimidine analogues (e.g. ancitabine, azacitidine, 6-azauridine, carmofur, cytarabine, dideoxyuridine, doxifluridine, enocitabine, floxuridine); androgens (e.g. calusterone, dromostanolone propionate, epitiostanol, mepitiostane, testolactone); anti-adrenals (e.g. aminoglutethimide, mitotane, trilostane); immune checkpoint inhibitors (e.g. the PD-1/PDL-1 interaction inhibitors BMS-1001 and BMS-1166); immune response modifiers (e.g. imiquimod and resiquimod); and pharmaceutically acceptable salts, derivatives or analogues of any of these compounds.

15. A method as claimed in claim 14, wherein the chemotherapeutic agent is an anti-metabolite, e.g. 5-fluorouracil, 5-fluorouridine, or gemcitabine.

16. A method as claimed in any one of claims 13 to 15, wherein said sonosensitising agent is selected from the group consisting of phenothiazine dyes (e.g. methylene blue, toluidine blue), Rose Bengal, porphyrins (e.g. Photofrin®), chlorins, benzochlorins, phthalocyanines, napthalocyanines, porphycenes, cyanines (e.g. Merocyanine 540 and indocyanine green), azodipyromethines (e.g. BODIPY and halogenated derivatives thereof), acridine dyes, purpurins, pheophorbides, verdins, psoralens, hematoporphyrins, protoporphyrins and curcumins.

17. A method as claimed in any one of the preceding claims which further comprises the step of loading a hydrophobic chemotherapeutic agent into the hydrophobic tail region of the lipid, preferably wherein said hydrophobic chemotherapeutic agent is an anti-microtubule agent, e.g. a taxol such as paclitaxel (PTX).

18. A method as claimed in any one of the preceding claims, wherein the microbubble comprises a shell which retains a gas selected from perfluorobutane, perfluoropropane, and oxygen.

19. A method as claimed in any one of the preceding claims, wherein the microbubble has a diameter in the range of from 0.05 to 100 μm.

20. A microbubble-therapeutic agent complex obtained or obtainable by a method as claimed in any one of the preceding claims.

21. A microbubble-therapeutic agent complex which comprises a microbubble shell formed from a plurality of lipids (e.g. from a plurality of phospholipids), wherein at least a proportion of said lipids are covalently linked to at least one therapeutic agent.

22. A pharmaceutical composition comprising a microbubble-therapeutic agent complex as claimed in claim 20 or claim 21, together with at least one pharmaceutical carrier or excipient.

23. A microbubble-therapeutic agent complex or a pharmaceutical composition as claimed in any one of claims 20 to 22 for use as a medicament.

24. A microbubble-therapeutic agent complex or a pharmaceutical composition as claimed in any one of claims 20 to 22 for use in a method of sonodynamic therapy, preferably for use in a method of combined sonodynamic therapy and chemotherapy.

25. A microbubble-therapeutic agent complex or a pharmaceutical composition for use as claimed in claim 24 in the treatment of cancer, metastasis or micrometastasis derived from said cancer, or in the treatment of circulating tumour cells (CTCs), preferably in the treatment of a deep-sited tumour such as pancreatic cancer.
